(19) United States
(12) Patent Application Publication
(10) Pub. No.: US 2025/0313875 A1
(43) Pub. Date: Oct. 9, 2025

(54) COMBINED FERMENTATION PROCESS FOR PRODUCING ONE OR MORE HUMAN MILK OLIGOSACCHARIDE(S) (HMO(S))

(71) Applicant: DSM IP Assets B.V., HEERLEN (NL)

(72) Inventors: Natalia CHRAPKOVA, Hørsholm (DK); Mikael JANSEN, Hørsholm (DK)

(21) Appl. No.: 18/708,983

(22) PCT Filed: Nov. 10, 2022
(86) PCT No.: PCT/EP2022/081516

§ 371 Date: May 9, 2024

(30) Foreign Application Priority Data
Nov. 11, 2021 (DK) PA202170552

(51) Int. Cl. C12P 19/04; C12P 19/02

(52) U.S. Cl. CPC C12P 19/04; C12P 19/02

(57) ABSTRACT

The present invention relates to the field of recombinant production of biological molecules in genetically modified cells. More particularly, it relates to a method for recombinant production of human milk oligosaccharides (HMO) using an improved seed fermentation process.

### CROSS-REFERENCE TO RELATED APPLICATIONS

This application is a national stage entry pursuant to 35 U.S.C. § 371 of International Application No. PCT/EP2022/081516, filed on Nov. 10, 2022, which claims priority to Denmark Application No. PA 202170552, filed on Nov. 11, 2021, the entire contents of all of which are hereby incorporated by reference in their entirety.

### FIELD

The present invention relates to the field of recombinant production of biological molecules in genetically modified cells. More particularly, it relates to a method for recombinant production of human milk oligosaccharides (HMO) using a new and improved fermentation process combining a batch and fed-batch fermentation mode in a seed fermentation step with a fed-batch and/or batch fermentation mode in a main/primary fermentation step.

### BACKGROUND

Human milk oligosaccharides (HMOs) are non-digestible carbohydrates and constitute the third largest component of mother's milk. No other mammal has a similar concentration or complexity of non-digestible oligosaccharides compared to human mother's milk. To date, more than 200 HMO's have been identified (see XI Chen, Chapter 4 of Advances in Carbohydrate Chemistry and Biochemistry, 2015, Volume 72 and Urashima et al.: Milk Oligosaccharides, Nova Biomedical Books, New York, 2011, ISBN: 978-1-61122-831-1).

HMOs have become of great interest in the last decade, due to the discovery of their important functionality in human development. Besides their prebiotic properties, HMOs have been linked to additional positive effects, which expands their field of application (Kunz C. et al., (2014) Food Oligosaccharides: Production, Analysis and Bioactivity, 1st Edition, p 5-20, Eds. Moreno J. and Luz Sanz M., John Wiley & Sons, Ltd). The health benefits of HMOs have enabled their approval for use in foods, such as infant formulas and foods, and for consumer health products.

HMOs can be synthesised chemically, this however poses a challenge in terms of producing large-scale quantities. To overcome the challenges associated with the chemical synthesis of HMOs, several enzymatic methods and fermentative approaches have been developed. Fermentation based processes have been developed for several HMOs, such as 2′-fucosyllactose, 3-fucosyllactose, lacto-N-tetraose, lacto-N-neotetraose, 3-sialyllactose and 6′-sialyllactose. Fermentation based processes typically utilize genetically modified bacterial strains, such as recombinant Escherichia coli (E. coli) (for review see Bych et al, Current Opinion in Biotechnology 2019, 56: 130-137).

Alongside the fermentative substrate and concentration, the yield and productivity of the HMO of interest are key parameters and major factors in determining the final production cost of HMO production. The main obstacles to effective fermentation are in general the use of food resources, inhibitory compounds released during biomass growth, substrate inhibition, large scale robustness, decreased product yield and productivity, inefficient utilization of carbon sources, and end product inhibition.

### SUMMARY

The present application describes an improved HMO fermentation process with an enhanced amount of product produced and/or a decreased time of fermentation, the process is particularly useful for large scale fermentation. The described process makes use of a combination of a batch and a fed-batch mode in the initial/seed fermentation of HMO producing microorganisms, which results in decreased the acetic acid production in the seed fermentation and increased biomass at the start of the main fermentation producing the HMO(s). The combination of batch and fed-batch mode in the seed step of the fermentation process surprisingly leads to HMO formation in the primary/main bioreactor being typically increased by at least 10-60% as compared to HMO formation in the primary/main bioreactor when the seed step is batch mode only.

The present invention relates to a new fermentation process for producing one or more Human Milk Oligosaccharide(s) (HMO(s)) comprising, providing a seed bioreactor with one or more feed lines, where the bioreactor is filled with a liquid medium comprising a low amount of carbon source/kg of medium, such as no more than 5-40 g, such as 10-20 g, such as no more than 13 g of a carbon source/kg of medium, inoculating the seed bioreactor with a HMO producing microorganism, to form a culture of HMO producing microorganisms, operating the seed bioreactor at conditions to promote growth of the microorganism(s) while continuously feeding to said seed bioreactor a medium with one or more carbon source(s), providing a primary bioreactor comprising a liquid medium, preferably with no added carbon source(s), passing at least a portion of the microorganism culture from the seed bioreactor, into the primary bioreactor, operating the primary bioreactor at conditions to promote growth of said microorganism(s) and to promote HMO production from said microorganism(s) while continuously feeding to said primary bioreactor a medium with one or more carbon source(s) and continuously adding to the primary bioreactor a substrate, such as lactose, fermenting the added carbon source(s) and substrate to produce a fermentation broth comprising a HMO producing microorganism(s) and one or more HMO product(s) and optionally, harvesting and/or purifying the one or more HMO(s) from the fermentation broth in the primary bioreactor/fermenter.

Typically, HMO formation in the primary bioreactor/fermenter is increased by at least 10% when using the seed culture generated with a fed-batch mode for seeding the primary bioreactor, compared to using an un-feed batch culture to seed the primary bioreactor.

It is currently envisioned that the one or more produced HMO is/are selected from the group consisting of LNT-II, pLNnH, LNT, LNnT, LNFP-1, LNFP-II, LNFP-III, LNFP-V, LNFP-VI, LNDFH-I, LNDFH-II, LNDFH-III, 2′-FL, DFL, 3FL, LST-a, 3′SL, 6′SL, LST-b, LST-c, FSL, FLST-a, DSLNT, LNnH and LNH.

In one embodiment, the continuous feeding of the seed reactor is initiated when the carbon source added and/or present in the start medium of the seed bioreactor is close to depletion (at the end of the initial batch phase).

In one embodiment, the feeding medium used in the seed fermentation process according to the present invention does not contain lactose.

In one embodiment, the continuously feeding to the seed bioreactor of one or more carbon source(s) results in a reduced acetic acid formation in the fermentation broth, such as below 250 mg/L, such as below 100 mg/L, such as between 60 and 80 mg/L in the seed culture at the end of fermentation, as measured by capillary electrophoresis.

In one embodiment, the primary/main bioreactor is initially fed with between 200 and 800 kg/h of one or more selected carbon source(s). The one or more carbon source(s) can be selected from the group consisting of glycerol, glucose, sucrose and mixtures thereof.

The HMO producing microorganism can be selected form the group consisting of Escherichia coli, Saccharomyces cerevisiae, Yarrowia lipolytica, Schizosaccharomyces pombe, Pichia pastoris, Kluveromyces lactis and Kluveromyces marxianus.

In the process described herein, the HMO producing microorganism expresses one or more protein(s) enabling the production of one or more HMO(s) in said cell and the expression of said protein(s) is/are controlled by one or more genetic regulatory element(s). In a presently preferred embodiment, the genetic regulatory element(s) regulates the expression of said protein through the carbon source(s) concentration, such that expression increases at low glucose or sucrose levels. The preferred one or more regulatory element(s) comprises a Plac and/or PglpF promoter sequence, and/or one or more functional variant(s) thereof

The seed bioreactor and/or the primary bioreactor of the fermentation process according to the present invention comprises one or more control units such as but not limited to temperature control unit, aeration control unit, growth rate control unit, biomass control unit, acetic acid control unit, feed rate control unit, titer rate control unit, overpressure control unit and/or pH control unit.

Other aspects and advantageous features of the present invention are described in detail and illustrated by non-limiting working examples below.

### FIGURES

FIG. 1. Fermentation process flow diagram showing the three main stages of current fermentation process for (A) standard batch mode seed fermentation and (B) combined batch and fed-batch seed fermentation (Superseed).

FIG. 2 (A) Relative glucose flow in percentage in the combined batch and fed-batch-seed fermentation (solid black line) and theoretical three different feeding profiles that could be applied for faster biomass development. (B) Expected biomass development in the combined batch and fed-batch seed fermentation (solid back line) and theoretical three different profiles that could be applied with corresponding biomass development in dashed lines.

FIG. 3 shows acetic acid (mg/L) development in seed fermentations showing the conventional batch seed fermentation (Standard seed dashed line with full circle)) and four different batches performed with the combined batch and fed-batch fermentation process (Superseed—solid lines) FIG. 4 shows OD development in seed fermentations showing the conventional batch seed fermentation (Standard seed dashed line with full circle) and four combined batch and fed-batch fermentation process (Superseed—solid lines) and the expected OD development in a combined batch and fed-batch fermentation process fermentation process (dotted line).

FIG. 5 shows biomass development in seed fermentation showing the conventional batch seed fermentation (Standard seed dashed line with full circle) and four combined batch and fed-batch fermentation process (Superseed—solid lines). Biomass is calculated as OD multiplied by the volume in kg's of the tank.

FIG. 6 shows relative glucose flow in percentage in main fermentation seeding with biomass from i) the conventional batch seed process (closed circles) or ii) the combined batch and fed-batch seed fermentation process (closed triangles).

FIG. 7 shows relative overall production rate in percentage, where the black bars correspond to the batches run with the conventional batch seed fermentation process, and hatched bars corresponding to the batches run with the combined batch and fed-batch seed fermentation process for 2′-FL with MP2.

FIG. 8 (A) OD development (B) Biomass development and (C) acetic acid development for combined batch and fed-batch seed fermentation for batch 2FL140 run with MP1.

FIG. 9 Acetic acid (mg/L) development in seed fermentations showing the conventional batch seed fermentation (standard seed, dashed line with full circles), four batches of the combined batch and fed-batch seed fermentations for MP2 and one batch of the combined batch and fed-batch seed fermentation for MP1 (full line with asterisk).

FIG. 10 OD development in seed fermentations showing the conventional batch seed fermentation (standard seed, dashed line with full circles), four batches of the combined batch and fed-batch seed fermentations for MP2 and one batch of the combined batch and fed-batch seed fermentation for MP1 (full line with asterisk).

FIG. 11 Biomass development in seed fermentation showing the conventional batch seed fermentation (standard seed, dashed line with full circles), four batches of the combined batch and fed-batch seed fermentations for MP2 and one batch of the combined batch and fed-batch seed fermentation for MP1 (full line with asterisk). Biomass is calculated as OD multiplied by the volume (kg) of the tank.

FIG. 12 shows the overall relative production rate for 3 batches using the conventional batch seed fermentation (black bars), and the combined batch and fed-batch seed fermentation for two batches of 2′-FL fermentation using the MP1 strain grown on glucose (hatched bars).

FIG. 13 Relative sucrose flow in percentage in main fermentation using the conventional batch seed process (solid circles) and the combined batch and fed-batch seed fermentation for MP3 (solid triangles).

FIG. 14 Relative overall production rate in percentage, where the solid bars correspond to the batches run with conventional batch seed fermentation process, and the hatched bars correspond to the batches run with the combined batch and fed-batch seed fermentation for LNnT with MP3.

FIG. 15 shows (A) LNT2 and (B) p-LNnH concentration relative to LNnT in percentage before and after implementation of the combined batch and fed-batch fermentation process.

### DETAILED DESCRIPTION

The current innovation relates to an improved HMO fermentation process with an enhanced amount of product produced and a decreased time of fermentation necessary to complete the main fermentation phase. The current innovation in particular relates to the application of a combined batch and fed-batch mode in the initial phase of fermentation, i.e., the seed fermentation, which takes place in a seed bioreactor, to decrease the acetic acid production in the seed fermenter, to improve viability of the HMO producing microorganisms and/or to increase biomass in the culture of HMO producing microorganisms which will be used to inoculate the main fermentation.

A conventional batch mode during seed fermentation typically uses between 25 to 40 g 100% glucose/kg medium, such as between 30 and 35 g/kg, such as 31 g 100% glucose/kg medium. Many bacteria, such as but not limited to E. coli, produce acetate during growth which they can reassimilate, however if they go into overflow metabolism the acetic acid starts accumulating in the bioreactor. The high carbon source (glucose) concentration at the beginning of the batch mode fermentation allows for a high growth rate of the microorganism, which may cause overflow metabolism in bacteria which can result in high acetic acid formation and/or oxygen limitation which eventually will lead to inhibited growth and cell death if continued. In other microorganisms such as yeast the overflow metabolism leads to ethanol formation which potentially have similar inhibitory effects as the acetic acid.

In embodiments the combined batch and fed-batch fermentation process in the seed bioreactor reduces the concentration of overflow metabolites such as acetic acid (acetate), lactic acid (lactate), formic acid (formate) and/or ethanol. Preferably overflow metabolites which have an inhibitory effect of cell growth are reduced.

E. coli in particular, is known to produce a significant amount of acetic acid when the growth rate exceeds 50% of the maximum growth rate, which is generally the case when cultured in a batch mode. Batch fermentation has well-known limitations with regards to the biomass production since, in particular for E. coli, the concentration of acetic acid in the culture and/or medium becomes toxic and affects the growth of the microorganism. As is shown in the experimental section, the fed-batch operation acts as an extension of the original batch seed fermentation phase. In the currently disclosed fermentation process, the seed fermentation process starts as a batch process but with a very low amount of carbon source. Once the initial carbon source has been consumed or is close to depletion, a continuous carbon feed is supplied to further increase the biomass to the desired level in a controlled manner.

In one embodiment, the carbon feed is started manually by the operators when the pH increases, which signalizes that the bacteria has consumed all the carbon source and has started consuming the organic acids. At the same time, the percentage dissolved oxygen (DO %) increases as the demand for oxygen becomes lower due to an overall drop in activity, as there is no or little carbon source available for the growth.

Due to the lower amounts of and more controlled access to the carbon source involved in the batch phase, less acetic acid is produced prior to the feeding as well as at the end of fermentation. This also leads to a better utilization of the carbon source. Thus, as a rule, in a properly controlled fed-batch seed fermentation, most of the carbon source is used for growth instead of on acetic acid production.

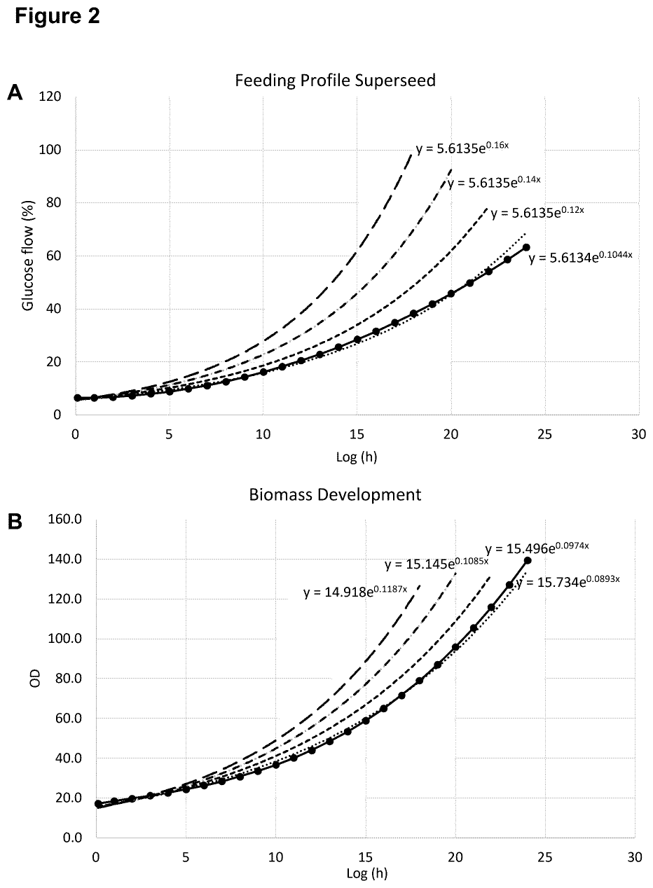

The relative glucose feeding profile utilized in the herein disclosed novel fermentation process is shown in FIG. 2A.

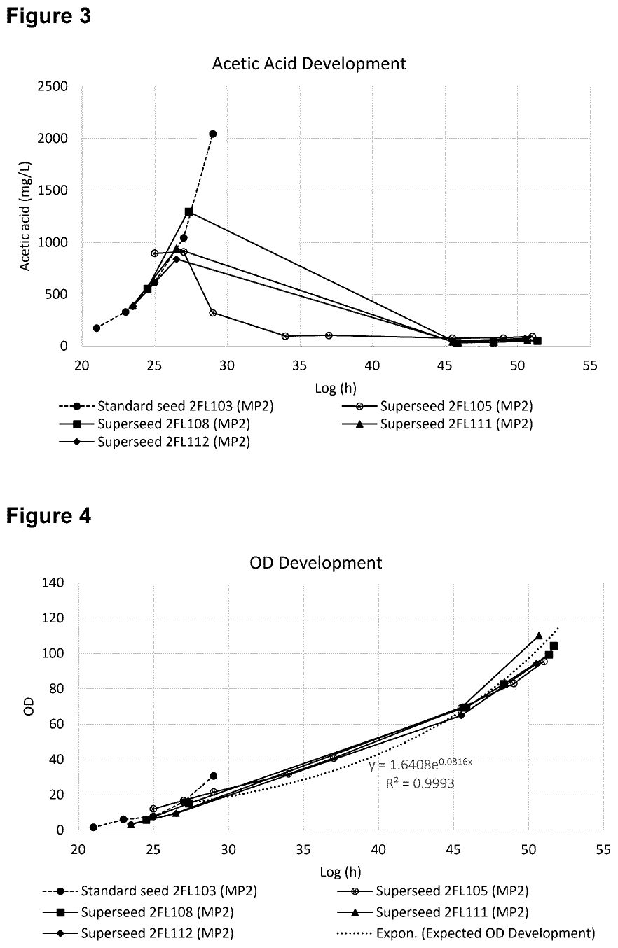

A comparison between a traditional batch-seed process and the herein for the first-time disclosed combined batch and fed-batch mode fermentation process, in terms of acetic acid and OD development, is displayed in FIG. 3 and FIG. 4, respectively. In particular, as a result of better controlled growth through continuous feed, the new fermentation process results in approximately up to 30 times reduced acetic acid and approximately up to 3 times higher biomass in comparison to the traditional batch-seed fermentation process. Furthermore, as FIG. 4 reveals, the OD development in all batches shows that the new fermentation process is a well-established and controlled process.

In embodiments the continuous feeding results in an acetic acid level at the end of fermentation that is 30 to 40 times lower than in the conventional batch seed fermentation.

In further embodiments the continuous feeding results in a volume that is at least 20% larger than what can be achieved with a batch fermentation. Furthermore, the continuous feeding results in a 3 to 5 times increase in biomass/L compared to the conventional batch seed fermentation. Therefore, the combined batch and fed-batch seed fermentation can be used to seed two production fermenters, instead of just one as done with the conventional batch seed fermentation. This essentially allows the operation of two or more main fermenters at the same time, with only one seed fermenter available to seed them, thereby increasing production efficiency even further.

As is clearly demonstrated in the experimental section, application of the combined batch and fed-batch seed fermentation process decreases the acetic acid production in seed fermentation, allowing the cells to enter the main/primary fermentation in a better condition, since accumulation of acetic acid can stress the cells and have a negative impact on the growth. Furthermore, the new fermentation process described herein can increase the productivity in the main fermentation at least in part due to higher biomass entering the main fermenter. The higher biomass allows application of increased carbon source feed in the main fermentation, thus reaching the area of high productivity faster. The increased productivity ultimately leads to an increased capacity of the fermentation.

Thus, the new fermentation process disclosed herein provides controlled growth of bacteria through fed-batch mode, thus preventing the accumulation of acetic acid and avoiding the negative impact it can have, as is shown in FIG. 3. The higher biomass originating from the novel seed fermentation process can be utilized in the main fermenter to reach higher productivity and high yield faster.

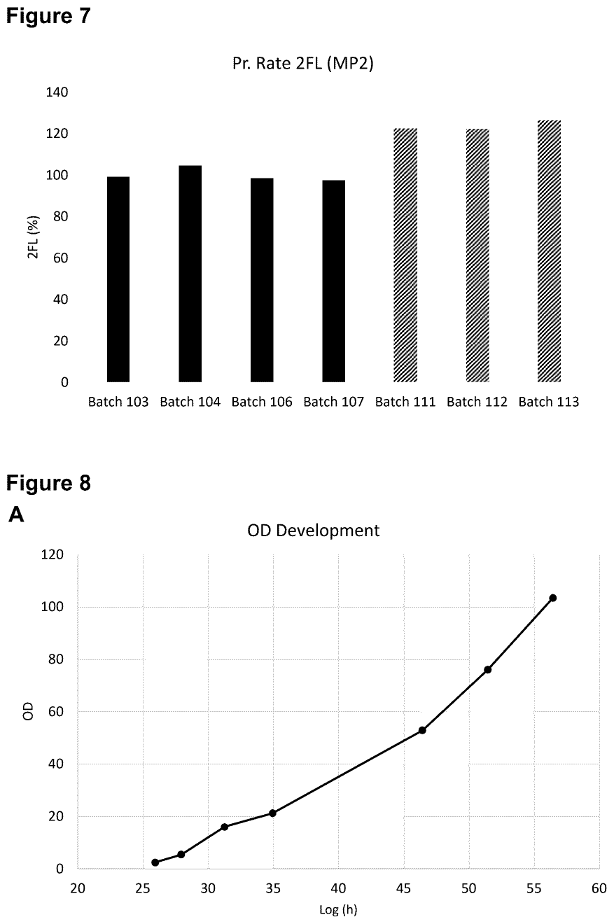

As FIG. 6 reveals, the new fermentation process allows using a higher carbon source feed at the beginning of the main/primary fermentation, which allows higher overall productivity in the main/primary fermenter. In one embodiment, the primary bioreactor is initially fed with between 200 and 800 kg/h of one or more selected carbon source(s), such as between 250 and 700 kg/h of one or more selected carbon source(s). As demonstrated in the experimental section, this leads to an average increase of at least 20% in overall productivity of the main fermenter. As is shown in FIG. 7, the batches produced with the novel seed fermentation process resulted on average in 20% increase in overall productivity for MP2 and MP1 strains, and in 60% increase in productivity for MP3 (FIG. 14). Without wanting to be limited by a scientific theory, the significant improvement in productivity could be attributed to reaching the area of high productivity faster, and thus extending the period of fermentation that is run under carbon limitation. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

In addition, the biomass could be increased further under different experimental settings. Essentially, the unexpected increase in biomass production in the initial seed fermentation could allow for seeding a plurality of main/primary fermentation reactions simultaneously, thereby increasing production efficiency even further.

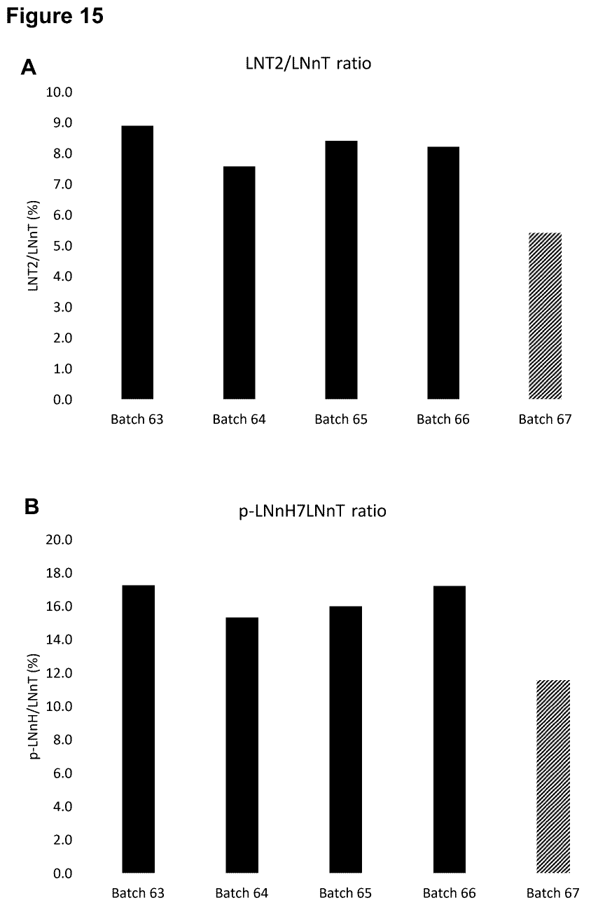

A further advantage is observed in in LNnT fermentation, where the two most abundant side products in LNnT fermentation, para-lacto-N-neohexaose (p-LNnH) and lacto-N-triose II (LNT2), are reduced. For an LNnT product, it is accepted that the side-products p-LNnH and LNT2 constitute 20% and 9% respectively of the total HMO. It is however desired to end the fermentation with as low side-product ratio as possible. As revealed in FIGS. 15A and B, the implementation of the herein described fermentation process resulted in a significant decrease in side-products with 30% decrease in p-LNnH and 35% decrease in LNT2 ratio relative to LNnT compared to traditional LNT production without the herein described combined batch and fad-batch seeding step.

In the following, embodiments of the invention will be described in further detail. Each specific variation of the features can be applied to other embodiments of the invention unless specifically stated otherwise.

Generally, all terms used herein are to be interpreted according to their ordinary meaning in the technical field, and applicable to all aspects and embodiments of the invention, unless explicitly defined or stated otherwise.

The terms “around”, “about” and “approximately” are used interchangeably and mean a 1-10% deviation of the indicated value, or a minor deviation that does not influence a relevant feature.

All references to “a/an/the [cell, sequence, gene, transporter, step, etc]” are to be interpreted openly as referring to at least one instance of said cell, sequence, gene, transporter, step, etc., unless explicitly stated otherwise.

The steps of any method disclosed herein do not have to be performed in the exact order disclosed, unless explicitly stated.

HMO(s) The present invention in general relates to a novel fermentation process for the efficient production of oligosaccharides. In particular, the present invention relates to a novel fermentation process which is employed to produce one or more HMO(s).

In the context of the invention, the term “oligosaccharide” means a saccharide polymer containing a number of monosaccharide units. In some embodiments, oligosaccharides are saccharide polymers consisting of three or four or five monosaccharide units, i.e., trisaccharides or tetrasaccharides or pentasaccharides. Preferable oligosaccharides of the invention are human milk oligosaccharides (HMOs).

The term “human milk oligosaccharide” or “HMO” in the present context means a complex carbohydrate found in human breast milk (for reference, see Urashima et al.: Milk Oligosaccharides. Nova Science Publisher (2011); or Chen, Adv. Carbohydr. Chem. Biochem. 72, 113 (2015)). The HMOs have a core structure comprising a lactose unit at the reducing end that can be elongated by one or more β-N-acetyl-lactosaminyl and/or one or more β-lacto-N-biosyl units, and this core structure can be substituted by an α-L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-triose 2 (LNT-2) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose Ill (LNFP-Ill), lacto-N-difucohexaose Ill (LNDFH-Ill), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH).

Examples of acidic and sialylated HMOs include 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In the context of the present invention lactose is not regarded as an HMO species.

In one preferred aspect of the invention, trisaccharide HMOs are produced.

In another preferred aspect of the invention, tetrasaccharide HMOs are produced.

### 2′-FL

2′-Fucosyllactose (2′-FL or 2′O-fucosyllactose) is a trisaccharide, more precisely, fucosylated, neutral trisaccharide composed of L-fucose, D-galactose, and D-glucose units (Fucα1-2Galβ1-4Glc). It is the most prevalent human milk oligosaccharide (HMO) naturally present in human breast milk, making up about 30% of all of HMOs. In a genetically modified cell or in an enzymatic reaction, 2′-FL is produced primarily by an α1,2-fucosyltransferase enzymatic reaction with lactose and a fucosyl doner.

### 3-FL

3-Fucosyllactose (3-FL) is a trisaccharide, more precisely, fucosylated, neutral trisaccharide composed of D-galactose, L-fucose and D-glucose (Galβ1-4(Fucα1-3)Glc). It is naturally present in human milk. In a genetically modified cell or in an enzymatic reaction, 3-FL is produced primarily by an α1,3-fucosyltransferase or α1,3/4-fucosyltransferase enzymatic reaction with lactose and a fucosyl doner.

### LNT

Lacto-N-tetraose (LNT) is a tetrasaccharide, more precisely, a neutral tetrasaccharide composed of galactose, N-acetylglucosamine, galactose, and glucose (GlcNAcβ1-3Galβ1-4Glc). It is naturally present in human milk.

### LNnT

Lacto-N-neotetraose (LNnT) is a tetrasaccharide, more precisely, a neutral tetrasaccharide composed of galactose, N-acetylglucosamine, galactose and glucose in a linear sequence, all joined by beta-linkages. (β-D-Gal-(1->3)-β-D-GlcNAc-(1->3)-β-D-Gal-(1->4)-D-Glc).

### DFL

Difucosyllactose (DFL or 2′,3-di-O-fucosyllactose) is an oligosaccharide, more precisely, fucosylated neutral tetrasaccharide composed of L-fucose, D-galactose, L-fucose, and D-glucose (Fucα1-2Galβ1-4(Fucα1-3)Glc). It is naturally present in human milk. In a genetically modified cell or in an enzymatic reaction, DFL is produced primarily by an α1,2-fucosyltransferase, α1,3-fucosyltransferase and/or α1,3/4-fucosyltransferase enzymatic reaction with lactose and two fucosyl doners.

### 3′-SL and 6′-SL

3′-Sialyllactose and 6′-Sialyllactose are oligosaccharides, more precisely, sialylated trisaccharides composed of N-acetylneuraminyl, galactose, and glucose (Neu5Ac-α2-3Galβ1-4-Glc or Neu5Ac-α2-6Galβ1-4-Glc). They are naturally occurring in human milk. The specific functional benefits of 3′-SL include reducing the risk of infection by inhibiting the adhesion of pathogenic bacteria e.g., Helicobacter pylori and their toxins or viruses e.g., Rotavirus. 3′-SL and 6′-SL in particular promote brain development in infants by supplying sialic acid, an essential building block for neurons.

### FSL (3′-S,3-FL)

3′-sialyl-3-fucosyllactose is an oligosaccharide, more precisely a sialylated and fucosylated tetrasaccharide composed of N-acetylneuraminic acid, D-galactose, L-fucose, and D-glucose units (Neu5Ac-α2-3Galβ1-4(Fucα1-3)Glc). It is naturally present in human milk.

### LST-a

Sialyllacto-N-tetraose A is an oligosaccharide, more precisely a sialylated pentasaccharide composed of N-acetylneuraminic acid, D-galactose, N-acetylglucosamine, D-galactose, and D-glucose units (Neu5Ac-α2-3Galβ1-3GlcNAcβ1-3Galβ1-4Glc). It is naturally present in human milk.

### LST-b

Sialyllacto-N-tetraose B is an oligosaccharide, more precisely a sialylated pentasaccharide composed of D-galactose, N-acetylneuraminic acid, N-acetylglucosamine, D-galactose, and D-glucose units (Galβ1-3(Neu5Ac-α2-6)GlcNAcβ1-3Galβ1-4Glc). It is naturally present in human milk.

### LST-c

Sialyllacto-N-neotetraose C is an oligosaccharide, more precisely a sialylated pentasaccharide composed of N-acetylneuraminic acid, D-galactose, N-acetylglucosamine, D-galactose, and D-glucose (Neu5Ac-α2-6Galβ1-4GlcNAcβ1-3Galβ1-4Glc). It is naturally present in human milk.

### DS-LNT

Disialyllacto-N-tetraose is an oligosaccharide, more precisely a sialylated hexasaccharide composed of N-acetylneuraminic acid, D-galactose, N-acetylneuraminic acid, N-acetylglucosamine, D-galactose, and D-glucose units (Neu5Ac-α2-3Galβ1-3(Neu5Ac-α2-6)GlcNAcβ1-3Galβ1-4Glc). It is naturally present in human milk.

### S-pLNnH

Sialyl-para-lacto-N-neohexaose is an oligosaccharide, more precisely a sialylated heptasaccharide composed of N-acetynleuraminic acid, D-galactose, N-acetylglucosamine, D-galactose, N-acetylglucosamine, D-galactose, and D-glucose units (Neu5Ac-α2-3Galβ1-4GlcNAcβ1-3Galβ1-4GlcNAcβ1-3Galβ1-4Glc.

### S-LNnH-I

Sialyl-lacto-N-neohexaose I is an oligosaccharide, more precisely a sialylated heptasaccharide composed of N-acetylneuraminic acid, D-galactose, N-acetylglucosamine, D-galactose, N-acetylglucosamine, D-galactose, and D-glucose units (Neu5Ac-α2-3Galβ1-4GlcNAcβ1-6(Galβ1-4GlcNAcβ1-3Galβ1-4Glc).

### DS-F-LNH II

Disialyl-fucosyl-lacto-N-hexaose II is an oligosaccharide, more precisely a sialylated, fucosylated nonasaccharide composed of N-acetylneuraminic acid, D-galactose, N-acetylglucosamine, N-acetylneuraminic acid, D-galactose, N-acetylglucosamine, L-fucose, D-galactose, and D-glucose units (Neu5Ac-α2-3Galβ1-3(Neu5Ac-α2-6)GlcNAcβ1-3(Galβ1-4(Fucα1-3)GlcNAcβ1-6)Galβ1-4Glc).

### FS-LNnH-I

Fucosyl-sialyl-lacto-N-neohexaose I is an oligosaccharide, more precisely a sialylated, fucosylated octasaccharide composed of N-acetylneuraminic acid, D-galactose, N-acetylglucosamine, D-galactose, L-fucose, N-acetylglucosamine, D-galactose, and D-glucose units (Neu5Ac-α2-6Galβ1-4GlcNAcβ1-3(Galβ1-4(Fucα1-3)GlcNAcβ1-6)Galβ1-4Glc).

### FS-LNH

Fucosyl-sialyl-lacto-N-hexaose is an oligosaccharide, more precisely a sialylated, fucosylated octasaccharide composed of N-acetylneuraminic acid, D-galactose, N-acetylglucosamine, L-fucose, D-galactose, N-acetylglucosamine, D-galactose, and D-glucose units (Neu5Ac-α2-6Galβ1-4GlcNAcβ1-6(Fucα1-2Galβ1-3GlcNAcβ1-3)Galβ1-4Glc).

### Fucosyl-LST-a (FLST-a)

Fucosyl-sialyllacto-N-tetraose A is an oligosaccharide, more precisely a sialylated, fucosylated hexasaccharide composed of N-acetylneuraminic acid, D-galactose, L-fucose, N-acetylglucosamine, D-galactose, and D-glucose units (Neu5Ac-α2-3Galβ1-3(Fucα1-4)GlcNAcβ1-3Galβ1-4Glc).

### Fucosyl-LST-b (FLST-b)

Fucosyl-sialyllacto-N-tetraose B is an oligosaccharide, more precisely a sialylated, fucosylated hexasaccharide composed of L-fucose, D-galactose, N-acetylneuraminic acid, N-acetylglucosamine, D-galactose, and D-glucose units (Fucα1-2Galβ1-3(Neu5Ac-α2-6)GlcNAcβ1-3Galβ1-4Glc).

### Fucosyl-LST-c (FLST-c)

Fucosyl-sialyllacto-N-neotetraose C is an oligosaccharide, more precisely a sialylated, fucosylated hexasaccharide composed of N-acetylneuraminic acid, D-galactose, N-acetylglucosamine, D-galactose, L-fucose, and D-glucose units (Neu5Ac-α2-6Galβ1-4GlcNAcβ1-3Galβ1-4(Fucα1-3)Glc.

### SLNH

Sialyl-lacto-N-hexaose is an oligosaccharide, more precisely a sialylated heptasaccharide composed of N-acetylneuraminic acid, D-galactose, N-acetylglucosamine, D-galactose, N-acetylglucosamine, D-galactose, and D-glucose units (Neu5Ac-α2-6Galβ1-4GlcNAcβ1-6(Galβ1-3GlcNAcβ1-3)Galβ1-4Glc).

### SLNnH-II

Sialyl-lacto-N-neohexaose II is an oligosaccharide, more precisely a sialylated heptasaccharide composed of N-acetylneuraminic acid, D-galactose, N-acetylglucosamine, D-galactose, N-acetylglucosamine, D-galactose, and D-glucose units (Neu5Ac-α2-6Galβ1-4GlcNAcβ1-3(Galβ1-4GlcNAcβ1-6)Galβ1-4Glc).

In a presently preferred embodiment, the herein disclosed fermentation process is used to produce one or more HMO(s) selected from the group consisting of LNT-II, pLNnH, LNT, LNnT, LNFP-1, LNFP-II, LNFP-III, LNFP-V, LNFP-VI, LNDFH-I, LNDFH-II, LNDFH-III, 2′-FL, DFL, 3FL, LST-a, 3′SL, 6′SL, LST-b, LST-c, FSL, FLST-a, DSLNT, LNnH and LNH.

In a presently preferred embodiment, the herein disclosed fermentation process is used to produce one or more HMO(s) selected from the group consisting of LNT, LNnT, 2′-FL, DFL, 3′SL, 6′SL and combinations thereof.

### Fermentation Process

The invention disclosed herein relates to an improved method for the production of one or more HMOs, the method comprising a new fermentation process with at least one seed bioreactor and at least one main/primary bioreactor for producing the HMO.

One aspect of the present invention is a method of providing a seed culture for an HMO fermentation process, in particular, for a large scale fermentation process, comprising a) providing a seed bioreactor with one or more feed lines, wherein the bioreactor contains a liquid medium comprising no more than 5-40 g of a carbon source/kg of medium, 10-20 g of a carbon source/kg of medium, such as no more than 13 g of a carbon source/kg of medium, b) inoculating the seed bioreactor with a HMO producing microorganism(s), c) operating the seed bioreactor at conditions to promote growth of the microorganism(s) by continuously feeding to said seed bioreactor a medium with one or more carbon source(s). Preferably, the continuously feeding is initiated when the initial carbon source in the medium is consumed or close to depletion. The continuous feeding can be maintained for as long as the space in the seed bioreactor allows or until sufficient biomass has been generated to seed one or more primary fermenter(s) for the HMO production.

In the context of the present invention large scale production is fermentations conducted in 20.000 L or more, such as 50.000 L, such as 100.000 L such as 200.000 L or more.

In practice, the herein disclosed fermentation process for production of one or more HMOs comprises at least one seed fermentation step (step a)-c)) and at least one main/primary fermentation step. Preferably, there is no product/HMO formation in the seed fermentation step whereas the primary fermentation step is conducted in a way that secures product formation.

Typically, HMO formation in the primary bioreactor is increased by at least 10%, such as by at least 20%, or even at least 60% when using the seed culture generated in step a)-c) for seeding the primary bioreactor of the fermentation process disclosed herein, compared to using a batch mode derived culture to seed the primary bioreactor.

### Combined Batch and Fed-Batch Mode

The fermentation process described herein comprises a seed-fermentation step that combines batch mode and fed-batch mode fermentation.

In the current context, the term “batch fermentation” is used to describe a process wherein all the substrate and nutrients are added at zero time or soon after inoculation takes place, and the vessel is allowed under a controlled environment to proceed until maximum end product concentration is achieved.

In batch fermentation, microorganisms are inoculated to a fixed volume of medium in a fermenter. With microbial growth, the nutrients are gradually consumed, and by-products accumulate. Therefore, the culture environment is continuously changing. The broth/fermentation medium is removed at the end of the run. The growth curve is usually divided into distinct phases. During the initial lag phase, growth is slow, as the organism needs to adapt to the new environment. During the exponential growth phase, the microbes divide at a constant rate. When nutrients are getting depleted and by-products accumulate, growth slows down, and the culture enters the stationary growth phase. At this point, the culture is usually harvested. If the culture continues, it will finally enter the death phase, which is characterized by a decrease in the viable cell density.

The advantages of batch processing are ease of operation and low risk of contamination. Disadvantages are the comparatively low cell densities which can be achieved. Furthermore, overflow metabolism may occur in batch fermentations due to the high initial amount of carbon-source in the culture medium resulting in high acetic acid formation, which is then transferred to the main bioreactor.

In contrast, the term “fed-batch fermentation”, “fed-batch mode” or fed-batch process”, herein used interchangeably, is used to describe a process wherein the nutrients are kept from becoming a limiting factor by constantly supplying them during cultivation. This is a partly open system.

Fed-batch fermentation is a modified version of batch fermentation. It is the most common mode of operation in production fermenters in the bioprocess industry. In the presently described combined batch and fed-batch process, microorganisms are inoculated and grown under batch regime for a certain amount of time, then nutrients are added to the fermenter in increments throughout the remaining duration of fermentation to feed them. The entire culture suspension is removed at the end of each run. The start of feeding is normally determined by carbon source limitation in the broth, and the time profile of feeding should be designed in a way that the carbon source remains non-excessive while microbial growth is fully supported.

Because of the addition of fresh nutrients, extensive biomass accumulation normally occurs in the exponential growth phase. Therefore, fed-batch fermentation is very useful for bioprocesses aiming for high biomass density or high product yield when the desired product is positively correlated with microbial growth. Also, because the carbon source is not overfed during the process, by-product accumulation is limited, in particular the formation of acetic acid in E. coli is limited.

A further type of fermentation used in the production fermenters in the bioprocess industry is the continuous fermentation, wherein fresh medium is continuously added to the fermenter, while used medium and cells are harvested at the same time. Consumed nutrients are replaced and toxic metabolites are removed from the culture. When addition and removal are at the same rate, the culture volume stays constant. Therefore, in contrast to fed-batch fermentation, the maximum working volume of the vessel does not limit the amount of fresh medium or feed solution which can be added to the culture in the course of the process. Keeping the working volume constant furthermore simplifies culture scale-up based on constant-power-to-volume strategy. The rate of medium exchange can be optimized to reach a steady state. In steady state, the cellular growth rate, and environmental conditions, like the concentrations of metabolites, stay constant. Cultures in steady state can last for days, weeks or even months, thus greatly reducing the downtime and making the process more economically competitive. Due to the long cultivation, sterility maintenance can be challenging, and downstream processing is complicated

While the batch process is classified as a discontinuous process, a fed-batch process is a semi-continuous process and/or a continuous process.

The herein disclosed fermentation process in one embodiment combines a batch and a fed-batch fermentation mode in the seed fermentation step with a fed-batch mode fermentation in the main/primary fermentation step. Alternatively, the primary/main fermentation can also be conducted in a batch fermentation mode, or in a continuous fermentation mode.

The present invention relates to a fermentation process for producing one or more Human Milk Oligosaccharide(s) (HMO(s)) comprising,
a) providing a seed bioreactor with one or more feed lines, wherein the bioreactor contains a liquid medium comprising no more than 5-40 g of a carbon source/kg of medium, such as no more than 13 g of a carbon source/kg of medium,
b) inoculating the seed bioreactor with an HMO producing microorganism(s),
c) operating the seed bioreactor at conditions to promote growth of the microorganism(s) by continuously feeding to said seed bioreactor a medium with one or more carbon source(s),
d) providing a primary bioreactor containing a liquid medium capable of supporting growth of the microorganism(s), preferably with no added carbon source(s),
e) passing at least a portion of the microorganism culture from the seed bioreactor, into the primary bioreactor,
f) operating the primary bioreactor at conditions to promote growth of said microorganism(s) and to promote HMO production from said microorganism(s) while continuously feeding to said primary bioreactor a medium with one or more carbon source(s) and continuously adding to the primary bioreactor a substrate for the HMO production, such as lactose or a fucosylated, sialylated or N-acetyl-glucosaminylated lactose trisaccharide,
g) fermenting the added carbon source(s) and substrate to produce a fermentation broth comprising the HMO producing microorganism(s) and one or more HMO product(s) and optionally,
h) harvesting and/or purifying the one or more HMO(s) from the fermentation broth.

In preferred embodiments the microorganism is Escherichia coli.

### Biorecators

In general, a bioreactor refers to any manufactured device or system that supports a biologically active environment. In one aspect, a bioreactor is a vessel in which a chemical process is carried out which involves microorganisms or biochemically active substances derived from such organisms. This process can either be aerobic or anaerobic. Bioreactors are commonly cylindrical, ranging in size from liters to cubic meters, or tons, and are often made of stainless steel or glass. In the current context, it refers to a device or system designed to grow cells or tissues in the context of a cell culture. The term bioreactor may be used interchangeably with the term fermenter.

### Seed Bioreactor

In the present context, the term “seed bioreactor” or “seed fermenter” are used interchangeably and encompasses a vessel suitable for fermenting a seed culture of a microorganism. The proportions of a seed bioreactor will depend on the size of production bioreactor it is meant to seed. Seed bioreactors are typically seeded with a culture from a T-flasks, roller bottles, shake flasks or a small-scale bioreactor system. Typically, a seed bioreactor for industrial scale production is a smaller version of the manufacturing bioreactor, the seed bioreactor is preferably at least 5 L, such as at least 20 L, such as at least 100 L, such as at least 1000 L, such as 10.000-50.000 L.

In one embodiment of the present invention, the seed bioreactor has a volume in the range of 10 to 50 tons, such as from 15 to 40 tons, such as from 20 to 35 tons, 27-32 tons, such as 20, 25, 27, 30 or 32 tons.

The purpose of the seeding fermentation is the generation of an adequate number of cells for the inoculation of a production bioreactor. Preferably, a seed fermentation does not produce any product, e.g., HMO.

### Main/Primary Bioreactor

In the current context, the term “main bioreactor” or “primary bioreactor” or “production bioreactor” or “main fermenter” or “primary fermenter” or “production fermenter” is used interchangeably and describes the bioreactor in which the microorganisms inoculated from the seed bioreactor are grown and in which the production of the HMO(s) by said microorganisms takes place.

Typically, the main/primary bioreactor used in the current fermentation process is a large-scale bioreactor which comprises a minimum volume of 5000 L, such as a minimum volume of 10.000 L, such as a minimum volume of 20.000 L such as a minimum volume of 50.000 L, such as a minimum volume of 100.000 L such as a minimum volume of 200.000 L, such as a volume in the range of 15.000-500.000 L.

In one embodiment of the present invention, the main bioreactor has a volume in the range of 20-500 tons, such as from 50-400 tons, such as from 80-350 tons.

### Liquid Medium

A growth medium, feed or culture medium is a liquid medium designed to support the growth of microorganisms, cells, or small plants. In the present context, the terms are used interchangeably.

A typical culture medium is composed of a complement of amino acids, vitamins, inorganic salts, a carbon source and serum as a source of growth factors, hormones, and attachment factors.

With regards to the suitable liquid medium used in the fermentation process, there are no limitations. The culture medium may be semi-defined, i.e., containing complex media compounds (e.g., yeast extract, soy peptone, casamino acids, etc.), or it may be chemically defined, without any complex compounds.

In the currently exemplified fermentation process, the seed fermentation basal medium is a fully defined minimal salt medium prepared with phosphoric acid (75%), KOH solution (45%), MgSO4, trace elements, citric acid, antifoam, and thiamine. The medium pH is controlled at 6.8 by sparging of gaseous NH3 together with the inlet air. Ammonia also serves as the only source of nitrogen. The trace elements include Mn, Cu, Fe, and Zn as sulfate salts. Lactose is not added.

### Carbon Source

In relation to the present invention, a carbon source is understood as the source of energy that the microorganism utilizes to build biomass.

Nutrients, such as carbon sources, may be continuously added to the fermenter, as in a fed-batch system, or may be charged into the reactor at the beginning of fermentation in a batch system.

In the fermentation process disclosed herein, the feeding of step c) and/or step f) comprises stepwise addition of one or more carbon source(s) and/or follows a linear and/or non-linear feeding profile of one or more carbon source(s), or a combination thereof.

In the currently exemplified fermentation process, the seed fermentation bioreactor is started by inoculation in basal medium, which is supplemented with in the range of 5-40 g carbon source/kg of medium, such as from 10-30 g/kg, such as from 12 to 20 g/kg. In one embodiment, the seed fermenter basal medium is supplemented with 12, 12.3, 12.5 or 13 g/kg of carbon source.

Once the carbon source in the basal medium is nearly consumed, a continuous feed of sterile carbon source solution is initiated (approximately 60% w/w) as the only source of carbon and energy at a pre-determined feeding profile (see FIG. 2A, solid line).

Preferably, the carbon source feed is started shortly before the end of batch phase to prevent full sugar depletion. In one embodiment, the carbon source feed is initiated manually by the operators when a carbon dioxide evolution rate (CER) of 45 was reached.

Alternatively, the feed can also be initiated when the pH starts to rise. pH measurement is done constantly in the bioreactor.

Typically, the carbon source feeding profile is designed to ensure carbon-limited conditions to avoid overflow metabolism and acetic acid formation.

Mineral salt levels are maintained by continuously feeding a separate co-feed solution to the fermenter, containing all the constituents of the basal medium.

Typically, the one or more carbon source(s) is/are selected from the group consisting of glycerol, glucose, sucrose and mixtures thereof. Alternative carbon sources can be selected from molasses, corn syrup, galactose, succinate, malate, pyruvate, lactate, ethanol, methanol, citrate and raffinose.

In one embodiment, the carbon source is glucose or sucrose and the seed bioreactor contains between 10-20 g of glucose or sucrose/kg of medium, such as between 10-15 g of glucose or sucrose/kg of medium, such as 12.3 g of glucose or sucrose/kg of medium, such as 12.5 g of glucose or sucrose/kg of medium, such as 13 g of glucose or sucrose/kg of medium, such as 14 g of glucose or sucrose/kg of medium. In the same embodiment, the feed continues until between 70 and 150 g of glucose or sucrose/kg of start medium has been added to the seed fermenter, between 85 and 125 g of glucose or sucrose/kg of start medium has been added to the seed fermenter, between 90 and 100 g of glucose or sucrose/kg of start medium has been added to the seed fermenter, such as 93 g of glucose or sucrose/kg of start medium has been added to the seed fermenter, such as between 95 g of glucose or sucrose/kg of start medium has been added to the seed fermenter, such as 98 g of glucose or sucrose/kg of start medium has been added to the seed fermenter. Due to the relatively low amount of sugar involved in the batch phase of the seed fermenter, and due to the continuous feeding of sugar in the fed-batch phase of the seed fermenter, less acetic acid is produced than in a conventional batch process.

The starting level of carbon source feeding can be determined by the biomass-specific sugar feed rate, also known as qₛ. In one embodiment, the starting qₛ is set to 60, which is a well-known level where the cells are able to grow and consume acetic acid at the same time. The carbon source feed at the start of feeding (t0) can be calculated using the below equation.

$$carbon {flow}_{{t}_{0}} = {q}_{S} \times {biomass}_{{t}_{0}}$$

$${biomass}_{{t}_{0}} = \frac{{OD}_{{t}_{0}} \times {V}_{{t}_{0}}}{{10}^{6}}$$

In one embodiment of the currently disclosed fermentation process, the feeding medium used in the seed fermenter does not contain lactose.

### One or More Feed Lines

For the fed-batch fermentation, nutrient feeding and medium harvest can be carried out following a pre-set time profile of pump speed. Alternatively, it is a common practice to rely on the percentage dissolved oxygen (DO) spike to signal fed-batch feeding start, however, the carbon source exhaustion that causes a DO spike may also shift the metabolism of the microorganism(s) and reduce peak biomass potential.

For continuous fermentation, two pumps can be assigned to follow the same time profile but with opposite directions, one for feeding, and the other one for harvest.

### Control Units

The environmental conditions inside the bioreactor, such as temperature, nutrient concentrations, pH, and dissolved gases (especially oxygen for aerobic fermentations) affect the growth and productivity of the organisms. The temperature of the fermentation medium can be maintained by a cooling jacket, coils, or both. Particularly exothermic fermentations may require the use of external heat exchangers.

The pH of the medium is measured and adjusted with small amounts of acid or base, depending upon the fermentation. For aerobic (and some anaerobic) fermentations, reactant gases (especially oxygen) must be added to the fermentation. Since oxygen is relatively insoluble in water (the basis of nearly all fermentation media), air (or purified oxygen) must be added continuously. The action of the rising bubbles helps mix the fermentation medium and also “strips” out waste gases, such as carbon dioxide. In practice, bioreactors are often pressurized, this increases the solubility of oxygen in water. In an aerobic process, optimal oxygen transfer is sometimes the rate limiting step. Oxygen is poorly soluble in water-even less in warm fermentation broths- and is relatively scarce in air (20.95%). Oxygen transfer is usually helped by agitation, which is also needed to mix nutrients and to keep the fermentation homogeneous. Gas dispersing agitators can be used to break up air bubbles and circulate them throughout the vessel.

The fermentation process according to the present invention comprises one or more control units selected from the group consisting of temperature control unit, aeration control unit, growth rate control unit, biomass control unit, acetic acid control unit, feed rate control unit, titer rate control unit, overpressure control unit and pH control unit.

During seed fermentation and main fermentation, samples are taken from the fermentation liquid in order to determine the concentration of organic acids such as acetic acid, formic acid and glutamic acid, e.g., using capillary electrophoresis.

Liquid medium samples are typically diluted in deionized water and boiled, followed by centrifugation, whereafter the resulting supernatant is analyzed by capillary electrophoresis.

### Operating the Seed Bioreactor

The seed bioreactor is operated at conditions to promote growth of the microorganisms while continuously feeding the seed bioreactor a medium with one or more carbon source(s). In one embodiment of the present invention, the seed fermentation comprises between 10 to 50 tons, such as from 15 to 40 tons, such as from 20 to 35 tons, 27-32 tons, such as 20, 25, 27, 30 or 32 tons.

Optical density measurement is used to determine the end of the seed fermentation phase, whereupon at least part of the microorganism culture of the seed bioreaction is used to inoculate the main/primary reaction. In embodiments the combined batch and batch-fed batch seed process is run for at least 30 hours, such as at least 35 hours, such as at least 40 hours, such as at least 45 hours, such as at least 50 hours to build as much biomass as possible while maintaining low acetate levels.

In the current context, at least 10%, such as at least 20, 30, 40, 50, 60, 70, 75, 80, 90, 95, 96, 97, 98, 99 or 100% of the microorganism culture of the seed bioreaction is used to inoculate the main/primary reaction. In a presently preferred embodiment, at least a part of, or the complete microorganism culture of the seed bioreaction is used to inoculate the main/primary bioreactor. In another embodiment at least a part of, or the complete microorganism culture of the seed bioreaction is used to inoculate more than one main/primary bioreactors, such as at least 2, 3 or 4 main bioreactors.

### Operating the Main/Primary Bioreactor

The primary/main bioreactor is operated at conditions to promote growth of said microorganism(s) and to promote HMO production from said microorganism(s) while continuously feeding to said primary bioreactor a medium with one or more carbon source(s) and continuously adding to the primary bioreactor a substrate, such as lactose.

The fermentation process according to the present invention is thus characterized by an increase of HMO formation in the primary bioreactor, when using the seed culture from the herein described combined batch and fed-batch seed process, compared to using a seed culture from a batch process to seed the primary bioreactor. Typically, HMO formation in the primary bioreactor is increased by at least 10% or at least 20%, such as by at least 15, 20, 25, 30, 35, 40, 45, 50, 60, 70, 80, 90 or 100%, when using the seed culture generated in step a)-c) for seeding the primary bioreactor (step e) compared to using a seed culture produced with a batch process to seed the primary bioreactor.

### Production of HMOs

To produce one or more HMOs, the HMO-producing microorganisms as described herein are cultivated in a primary/main bioreactor according to the procedures known in the art in the presence of a suitable carbon source, e.g., glucose, glycerol, sucrose and a suitable substrate for HMO production, such as lactose, or a fucosylated, sialylated or N-acetyl-glucosaminylated lactose trisaccharide or tetrasaccharide, such as, but not limited to 2′-FL, 3-FL, 3′-SL, 6′-SL, LNTII, LNT or LNnT, as described herein. The produced HMO is harvested from the cultivation media and the microbial biomass formed during the cultivation process. Preferably the HMOs are secreted from the cell into the culture medium.

Thereafter, the HMOs are purified according to the procedures known in the art (see purification of HMOs below).

Manufacturing of HMOs is typically accomplished by performing cultivation in larger volumes. The term “manufacturing” and “manufacturing scale” in the meaning of the invention defines a fermentation with a minimum seed culture volume of 10 L, such as 100 L, such as 1000 L, such as 2000 L and a primary fermentation volume of at least 100 L, such as 1000 L, such as 10.000 L, such as 20.000 L. An example of a large-scale HMO manufacture uses a seed culture volume of at least 5 tons, such as at least 9 tons to seed a primary fermentation of at least 50 tons, such as at least 90 tons. Generally, the seed fermenter volume is 1/10 of the manufacturing fermenter volume. Usually, a “manufacturing scale” process is defined by being capable of processing large volumes of a preparation containing the product of interest and yielding amounts of the product of interest that meet, e.g., in the case of a food compound/additive or therapeutic compound or composition, the demands for safety studies, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.).

By the term “one or more HMOs” is meant that an HMO microorganism may be able to produce a single HMO structure (a first HMO) or multiple/blends of HMO structures (a second, a third, etc. HMO). In some embodiments, it may be preferred a genetically modified microorganism that produces a single HMO (meaning exceeding 90%, such as 95%, such as 98% of the total HMOs produced), in other preferred embodiments, a genetically modified microorganism producing multiple HMO structures may be preferred. Non-limiting examples for genetically modified microorganisms producing single HMO structures are 2′-FL, 3-FL, 3′-SL, 6′-SL or LNT-2 producing microorganisms. Non-limiting examples of genetically modified microorganisms capable of producing multiple HMO structures can be DFL, FSL, LNT, LNnT, LNFP I, LNFP II, LNFP III, LNFP IV, LNFP V, pLNnH, pLNH2, LSTa, LSTb, LSTc, DSLNT, F-LSTa and F-LSTb producing microorganisms.

### Fermenting

According to the invention, the terms “culturing” (or “cultivating” or “cultivation”, also termed “fermentation”) relate to the propagation of a culture of microorganisms in a controlled bioreactor according to methods known in the industry.

The fermentation process in the current method is predominantly aerobic. The air flow in the primary bioreactor is in one embodiment in the range of 3000-19 000 Nm3/h.

### Reduced Acetic Acid Formation in the Fermentation Broth

The fermentation process according to the present invention comprises continuously feeding to the seed bioreactor or primary bioreactor of one or more carbon source(s), which results in a reduced acetic acid formation in the fermentation broth.

The continuously feeding to said seed bioreactor of one or more carbon source(s) typically results in a reduced acetic acid formation to below 250 mg/L, such as below 200, 100 or 80 mg/L, such as approximately between 60 and 80 mg/L, such as between 60 and 70 mg/L in the seed culture at the end of fermentation, as measured by capillary electrophoresis.

In one embodiment, the continuously feeding to said seed bioreactor of one or more carbon source(s) results in an acetic acid formation of approximately 70, 69, 68, 67 or 66 mg/L in the seed culture at the end of fermentation, as measured by capillary electrophoresis.

In another embodiment the continuously feeding to said primary bioreactor of one or more carbon source(s) results in an acetic acid formation below 500 mg/L, such as below 250 mg/L, such as between 50 and 500 mg/L in the primary culture at the end of fermentation, as measured by capillary electrophoresis.

### OD Development

In the current context, biomass is assessed by measuring optical density at OD600.

Optical density measurement is a well-known method to determine growth in a biofermenter. After DO sensor calibration and right before inoculation, 20 mL of fresh medium is taken from the vessel. One milliliter of medium is used to set blank for measurement of optical density at 600 nm on an Ultrospec 10 (biochrom), and the rest is used to dilute the dense microorganism suspension collected in the later phase during fermentation. Samples are taken every 0.5 to 1 hours until a decreasing trend of OD600 is observed. In one embodiment, a 10% sodium chloride (NaCl) solution can be used for dilution instead of the fresh medium.

As shown in the experimental section, the biomass is calculated as OD multiplied by volume of the tank in kilograms.

A comparison between a batch-seed process and the herein describe combined batch and fed-batch seed process in terms of OD and biomass development shows that the herein describe combined batch and fed-batch seed process results in more than 3 times higher, such as more than 4-, 5-, 6-, 7-, 8-, 9-, or 10-times higher biomass in comparison to the batch-seed fermentation process.

The significant increase in biomass in the seed fermenter in turn leads to higher biomass inoculum of the main fermenter. Wherein a batch-seed fermenter starting with 27 tons culture medium approximately delivers biomass of 7,9E+05, the new combined batch and fed-batch seed process delivers biomass of approximately 3,2E+06.

Harvesting the One or More HMO(s) from the Fermentation Broth

The term “harvesting” in the context in the invention relates to collecting the produced HMO(s) following the termination of fermentation. In different embodiments it may include collecting the HMO(s) included in both the biomass (i.e., the cells of the microorganisms) and cultivation media, i.e., before/without separation of the fermentation broth from the biomass. In other embodiments the produced HMOs may be collected separately from the biomass and fermentation broth, i.e., after/following the separation of biomass from cultivation media (i.e., fermentation broth). The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation) includes extraction thereof from the biomass (the production cells). It can be done by any suitable methods of the art, e.g., by sonication, boiling, homogenization, enzymatic lysis using lysozyme, or freezing and grinding.

After recovery from fermentation, HMO(s) are available for further processing and purification.

### HMO Producing Microorganisms

The fermentation process according to the present invention comprises cultivating HMO producing microorganisms which expresses one or more protein(s) enabling the production of one or more HMO(s) in said microorganism and wherein the expression of said protein(s) is/are controlled by one or more genetic regulatory element(s). Preferably, this regulatory element enables expression of said protein(s) when the carbon source(s) is maintained at carbon-limited conditions.

Thus, the fermentation process according to the present invention comprises cultivating genetically modified microorganisms.

A “genetically modified microorganism” as used herein is understood as a microorganism where the genetic material has been altered by human intervention using a genetic engineering technique, such a technique is for example, but not limited to, transformation or transfection e.g., with a heterologous polynucleotide sequence, Crisper/Cas editing and/or random mutagenesis. In the present context, the terms a“genetically modified microorganism” and “a host cell” and “a host microorganism” are used interchangeably.

In the present invention the “genetically modified microorganism” is preferably a host microorganism which has been transformed or transfected by an exogenous polynucleotide sequence. Preferably the polynucleotide sequence is genomically integrated.

The genetically modified microorganism is preferably a prokaryotic microorganism. Appropriate microbial microorganisms that may function as a host microorganism include yeast microorganisms, bacterial microorganisms, archaebacterial microorganisms, algae microorganisms, and fungal microorganisms.

The genetically modified microorganism (host microorganism or recombinant microorganism) may be e.g., a bacterial or yeast microorganism. In one preferred embodiment, the genetically modified microorganism is a bacterial microorganism.

Regarding the bacterial host microorganisms, there are, in principle, no limitations, they may be eubacteria (gram-positive or gram-negative) or archaebacteria, as long as they allow genetic manipulation for insertion of a gene of interest and can be cultivated on a manufacturing scale. Preferably, the host microorganism has the property to allow cultivation to high microorganism densities. Non-limiting examples of bacterial host microorganisms that are suitable for recombinant industrial production of an HMO(s) according to the invention could be Escherichia coli, Erwinia herbicola (Pantoea agglomerans), Citrobacter freundii, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be modified using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species for the invention described herein. Also included as part of this invention are strains, modified as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Bacteria comprising the characteristics described herein are cultured in the presence of lactose, and an oligosaccharide, such as an HMO, produced by the microorganism is retrieved, either from the bacterium itself or from a culture supernatant of the bacterium. In one preferred embodiment, the genetically modified microorganism of the invention is an Escherichia coli cell.

Non-limiting examples of fungal host cells that are suitable for recombinant industrial production of a heterologous product could be yeast cells, such as Komagataella phaffii, Kluyveromyces lactis, Yarrowia lipolytica, Pichia pastoris, and Saccaromyces cerevisiae or filamentous fungi such as Aspargillus sp, Fusarium sp or Thricoderma sp, exemplary species are A. niger, A. nidulans, A. oryzae, F. solani, F. graminearum and T. reesei.

In another preferred embodiment the host microorganism is a yeast cell e.g., Saccharomyces cerevisiae, Schizosaccharomyces pombe, Pichia pastoris, Komagataella phaffii, Yarrowia lipolytica, Kluveromyces lactis, Kluveromyces marxianus, etc.

Genetically modified microorganisms of the invention can be provided using standard methods of the art e.g., those described in the manuals by Sambrook et al., Wilson & Walker, “Maniatise et al., and Ausubel et al.

A fermentation process according to the present invention comprises culturing a microorganism which is preferably selected form the group consisting of Escherichia coli, Saccharomyces cerevisiae, Bacillus subtilis, Yarrowia lipolytica, Schizosaccharomyces pombe, Pichia pastoris, Kluveromyces lactis and Kluveromyces marxianus.

### Purification of HMOs

Purification of HMOs produced by fermentation can be done using a suitable procedure described in WO2016/095924, WO2015/188834, WO2017/152918, WO2017/182965, US2019/0119314 (all incorporated by reference).

HMO products of the invention as described are preferably 2′-FL, DFL, 2′-FL/DFL, 3′SL, 6′SL, LNnT, LNT, 3-FL, LNFP-1 and mixtures thereof.

### Functional Enzymes for HMO Synthesis

To be able to synthesize one or more HMOs, a recombinant microorganism needs to comprise at least one recombinant and/or heterologous nucleic acid which encodes a functional enzyme with glycosyltransferase activity. The glycosyltransferase gene may be integrated into the genome (by chromosomal integration) of the genetically modified microorganism, or alternatively, it may be comprised in a plasmid DNA and expressed as plasmid borne. If two or more glycosyltransferases are needed for the production of an HMO, e.g., for producing LNT or LNnT, two or more recombinant nucleic acids encoding different enzymes with glycosyltransferase activity may be integrated in the genome and/or expressed from a plasmid, e.g., a β-1,3-N-acetylglucosaminyltransferase (a first recombinant nucleic acid encoding a first glycosyltransferase) in combination with a β-1,3-galactosyltransferase (a second recombinant nucleic acid encoding a second glycosyltransferase) for the production of LNT, where the first and second recombinant nucleic acid can independently from each other be integrated chromosomally or on a plasmid.

In one preferred embodiment, both the first and second recombinant nucleic acids are stably integrated into the chromosome of the production microorganism; in another embodiment at least one of the first and second glycosyltransferase is plasmid-borne. A protein/enzyme with glycosyltransferase activity (glycosyltransferase) may be selected in different embodiments from enzymes having the activity of α-1,2-fucosyltransferase, α-1,3-fucosyltransferase, α-1,3/4-fucosyltransferase, α-1,4-fucosyltransferase α-2,3-sialyltransferase, α-2,6-sialyltransferase, β-1,3-N-acetylglucosaminyltransferase, β-1,6-N-acetylglucosaminyltransferase, β-1,3-galactosyltransferase and β-1,4-galactosyltransferase (see Faijes et al 2019 Biotechnology Advances 37; 667-697 or review of glycosyltransferase in HMO production). For example, the production of 2′-FL requires that the modified microorganism expresses an active α-1,2-fucosyltransferase enzyme. For the production of 3-FL, the modified microorganism needs expression of an active α-1,3-fucosyltransferase enzyme. For the production of LNT, the modified microorganism needs to express at least two glycosyltransferases, a β-1,3-N-acetylglucosaminyltransferase and a β-1,3-galactosyltransferase. For the production of 6′-SL, the modified microorganism has to express an active α-2,6-sialyltransferase enzyme and a pathway for generating a sialate sugar nucleotide, such as a pathway for CMP-sialic acid synthesis, in particular synthesis of CMP-neu5A. For the production of 3′-SL, the modified microorganism has to express an active α-2,3-sialyltransferase enzyme and a pathway for generating a sialate sugar nucleotide, such as a pathway for CMP-sialic acid synthesis, in particular in particular synthesis of CMP-neu5A.

Recombinant and/or Heterologous Nucleic Acid Sequences

By the term “heterologous nucleic acid sequence”, is meant a nucleic acid sequence in the host cell which is derived from a different organism or artificially produced.

By “recombinant gene/nucleic acid/DNA” is meant either a heterologous nucleic acid sequence or an endogenous nucleic acid sequence that has been manipulated, e.g., by introduction of an additional copy thereof or making mutations therein in the host cell. A recombinant nucleic sequence may be a coding DNA sequence, e.g., a gene, or non-coding DNA sequence, e.g., a regulatory DNA, such as a promoter sequence. One aspect of the invention relates to fermenting a recombinant microorganism comprising recombinant DNA sequences encoding enzymes necessary for the production of one or more HMOs.

“Coding or encoding nucleic acid sequence/gene/DNA” refers to a set of consecutive, non-overlapping triplets (codons) which is transcribed into mRNA and translated into a polypeptide when under the control of the appropriate control sequences, i.e., a promoter. Nucleic acid sequences can be produced artificially (i.e., produced in vitro using standard laboratory methods for making nucleic acid sequences) and inserted into the host cell. The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleotide sequences encoding a given protein may be produced. The term nucleic acid is used interchangeably with the term “polynucleotide”. An “oligonucleotide” is a short chain nucleic acid molecule.

The term “operably linked” refers to a functional relationship between two or more nucleic acid (e.g., DNA) segments. Typically, it refers to the functional relationship of a transcriptional regulatory sequence to a transcribed sequence. For example, a promoter sequence is operably linked to a coding sequence if it stimulates or modulates the transcription of the coding sequence in an appropriate host microorganism or other expression system. Generally, promoter transcriptional regulatory sequences that are operably linked to a transcribed sequence are physically contiguous to the transcribed sequence, i.e., they are cis-acting.

The term “nucleic acid construct” means an artificially constructed segment of nucleotides, in particular a DNA segment. In the context of generating an HMO producing host cell, the nucleic acid construct contains a recombinant DNA sequence comprising two or more recombinant DNA sequences: essentially, a non-coding DNA sequence comprising a promoter DNA sequence and one or more coding DNA sequence encoding a gene(s) of interest, e.g., a glycosyltransferase, or another gene useful for production of an HMO in a host cell. Preferably, the construct comprises further non-coding DNA sequences that either regulate transcription or translation of the coding DNA of the construct, e.g., a DNA sequence facilitating ribosome binding to the transcript, a leading DNA sequence that stabilize the transcript. The nucleic acid construct used to generate the host cell for HMO production may be a part of the vector DNA, or it may be part of an expression cassette/cartridge that is integrated in the genome of a host microorganism.

Integration of the recombinant nucleic acid of interest comprised in the construct (expression cassette) into the bacterial genome can be achieved by conventional methods, e.g., by using linear cartridges that contain flanking sequences homologous to a specific site on the chromosome, as described for the attTn7-site (Waddell C. S. and Craig N. L., Genes Dev. (1988) February; 2(2):137-49); methods for genomic integration of nucleic acid sequences in which recombination is mediated by the Red recombinase function of the phage A or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56; Vetcher et al., Appl. Environ Microbiol. (2005); 71(4):1829-35); or positive clones, i.e., clones that carry the expression cassette, can be selected e.g., by means of a marker gene, or loss or gain of gene function.

A single copy of the expression cassette comprising a gene of interest may be sufficient to secure production of a desired HMO and achieve the desired effects according to the invention. Accordingly, in some preferred embodiments, the invention relates to a recombinant HMO producing microorganism that comprises one, two or three copies of a gene of interest integrated in the genomic DNA of the microorganism. In some embodiments the single copy of the gene is preferred.

In a preferred embodiment the HMO producing microorganism (e.g., E. coli) comprises a sialic acid synthetic capability, i.e., the genetically modified microorganism comprises a biosynthetic pathway for making a sialate sugar nucleotide. For example, the genetically modified bacterium comprises a sialic acid synthetic capability through provision of an exogenous UDP-GlcNAc 2-epimerase (e.g., neuC of Campylobacter jejuni (GenBank AAK91727.1) or equivalent (e.g., (GenBank CAR04561.1), a Neu5Ac synthase (e.g., neuB of C. jejuni (GenBank AAK91726.1) or equivalent, (e.g., Flavobacterium limnosediminis sialic acid synthase, GenBank WP_023580510.1), and/or a CMP-Neu5Ac synthetase (e.g., neuA of C. jejuni (GenBank AAK91728.1) or equivalent, (e.g., Vibrio brasiliensis CMP-sialic acid synthase, GenBank WP_006881452.1).

Production of neutral N-acetylglucosamine-containing HMOs in modified bacteria is also known in the art (see e.g., Gebus C et al. (2012) Carbohydrate Research 363 83-90).

For the production of N-Acetylneuraminic acid (sialyl) containing HMOs, such as 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), sialyl-lacto-N-hexaose (SLNH), sialyl-lacto-N-neohexaose I (SLNH-1), sialyl-lacto-N-neohexaose II (SLNH-II) and disialyl-lacto-N-tetraose (DSLNT), sialyl-para-lacto-N-neohexaose (S-pLNnH), sialyl-lacto-N-neohexaose I (S-LNnH-1), disialyl-fucosyl-lacto-N-hexaose II (DS-F-LNH 1l), fucosyl-sialyl-lacto-N-neohexaose I (FS-LNnH-1) and/or fucosyl-sialyl-lacto-N-hexaose (FS-LNH) said genetically modified microorganism is modified to comprise an exogeneous N-Acetylneuraminic acid transferase (i.e., a sialyl transferase), or a functional variant or fragment thereof. An exogenous sialyl transferase gene may be obtained from any one of a number of sources.

For the production of N-acetylglucosamine-containing HMOs, such as Lacto-N-triose 2 (LNT-2), Lacto-N-tetraose (LNT), Lacto-N-neotetraose (LNnT), Lacto-N-fucopentaose I (LNFP-1), Lacto-N-fucopentaose II (LNFP-II), Lacto-N-fucopentaose Ill (LNFP-Ill), Lacto-N-fucopentaose V (LNFP-V), Lacto-N-difucohexaose I (LDFH-I), Lacto-N-difucohexaose II (LDFH-II), and Lacto-N-neodifucohexaose II (LNDFH-III), as described above, and it is modified to comprise an exogenous UDP-GlcNAc:Galα/β-R β-3-N-acetylglucosaminyltransferase gene, or a functional variant or fragment thereof. This exogenous UDP-GlcNAc:Galα/β-R β-3-N-acetylglucosaminyltransferase gene may be obtained from any one of a number of sources, e.g., the IgtA gene described from N. meningitides (Genbank protein Accession AAF42258.1) or N. gonorrhoeae (Genbank protein Accession ACF31229.1). Optionally, an additional exogenous glycosyltransferase gene may be co-expressed in the bacterium comprising an exogenous UDP-GlcNAc:Galα/β-R β-3-N-acetylglucosaminyltransferase. For example, a β-1,4-galactosyltransferase gene is co-expressed with the UDP-GlcNAc:Galα/β-R β-3-N-acetylglucosaminyltransferase gene. This exogenous β-1,4-galactosyltransferase gene can be obtained from any one of a number of sources, e.g., the one described from N. meningitidis, the IgtB gene (Genbank protein Accession AAF42257.1), or from H. pylori, the HP0826/galT gene (Genbank protein Accession NP_207619.1). Optionally, the additional exogenous glycosyltransferase gene co-expressed in the bacterium comprising an exogenous UDP-GlcNAc:Galα/β-R β-3-N-acetylglucosaminyltransferase gene is a β-1,3-galactosyltransferase gene, e.g., that described from E. coli 055:H7, the wbgO gene (Genbank protein Accession WP_000582563.1), or from H. pylori, the jhp0563 gene (Genbank protein Accession AEZ55696.1), or from Streptococcus agalactiae type Ib 012 the cps/BJ gene (Genbank protein Accession AB050723). Functional variants and fragments of any of the enzymes described above are also encompassed by the disclosed invention.

A sialyl transferase gene, N-acetylglucosaminyltransferase gene and/or a galactosyltransferase gene, can also be operably linked to a Pglp promotor and be expressed from the corresponding genome-integrated cassette. In one embodiment, the gene that is genomically integrated is a gene encoding for a galactosyltransferase, e.g., HP0826 gene encoding for the GalT enzyme from H. pylori (Genbank protein Accession NP_207619.1); in another embodiment, the gene that is genomically integrated is a gene encoding a β-1,3-N-acetylglucosaminyltransferase, e.g., IgtA gene from N. meningitidis (Genbank protein Accession AAF42258.1); In another embodiment, the gene that is genomically integrated is a gene encoding a α-2,3-sialyltransferase e.g., NST of Neisseria meningitidis (Genbank protein accession AAC44541.1). In these embodiments the gene encoding a β-1,3-N-acetylglucosaminyltransferase or galactosyltransferase, correspondingly, may either be expressed from a genome-integrated or plasmid borne cassette.

HMO producing host microorganisms typically comprise one or more polypeptide(s) capable of sugar transportation is a polypeptide belonging to the Major Facilitator Superfamily (MFS), such as those described in WO2021/148615, WO2021/148614, WO2021/14861 1, WO2021/148620, WO2021/148618 and WO2021/148610. HMO producing host microorganisms typically comprise a functional lacY and a dysfunctional lacZ gene.

### Regulatory Element

The genetically modified microorganism may further comprise a nucleic acid sequence comprising a regulatory element for the regulation of the expression of a recombinant and/or heterologous nucleic acid sequence. The nucleic acid sequence of the regulatory region may be heterologous or homologous. The regulatory element of the nucleic acid construct of the present invention is preferably selected from the group consisting of PglpF, PglpF_SD4 and PglpF_SD7. Examples of PglpF promoter sequences are described in WO2019/123324, hereby incorporated by reference, in particular, SEQ ID NO: 12, SEQ ID NO: 16 and SEQ ID NO: 19 of WO 2019/123324 are hereby incorporated by reference). Without being bound by theory, the use of the PglpF promoter and variants thereof may be particularly useful in relation to the present invention since a global transcription regulator formed under glucose limitation could be facilitating the genome-based stable controllable high-yield production.

In one embodiment, the fermentation process according to the present invention comprises culturing a microorganism further comprising a nucleic acid sequence comprising a regulatory element for the regulation of the expression of a nucleic acid sequence, wherein the and the one or more regulatory element(s) comprises a Plac and/or PglpF promoter sequence, and/or one or more functional variant(s) thereof. The original Plac promoter is described as SEQ ID NO: 11 in WO 2019/123324 (hereby incorporated by reference).

The term, a “regulatory element” or “promoter” or “promoter region” or “promoter element” is a nucleic acid sequence that is recognized and bound by a DNA dependent RNA polymerase during initiation of transcription. The promoter, together with other transcriptional and translational regulatory nucleic acid sequences (also termed “control sequences”) is necessary to express a given gene or group of genes (an operon). In general, the transcriptional and translational regulatory sequences include, but are not limited to, promoter sequences, ribosomal binding sites, transcriptional start and stop sequences, translational start and stop sequences, and enhancer or activator sequences. The “transcription start site” means the first nucleotide to be transcribed and is designated +1. Nucleotides downstream of the start site are numbered +2, +3, +4 etc., and nucleotides in the 5′ opposite (upstream) direction are numbered −1, −2, −3 etc. The promoter of the construct can derive from a promoter region of any gene encoded in the genome of a species. Preferably, a promoter region of the genomic DNA of E. coli. Accordingly, any promoter that is able to bind to an RNA polymerase and initiate transcription is suitable for practicing the invention. In principle, any promoter can be used to control transcription of the recombinant gene, such as the MFS transporter or the glycosyltransferases of the invention. In carrying out the invention, different or identical promoter sequences may be used to drive transcription of different genes of interest integrated in the genome of the host cell or in expression vector DNA. In one non-limiting example, promoter sequence A is promoting the expression of a MFS transporter, and another promoter sequence B or identical promoter sequence A is promoting the expression of a glycosyltransferase.

To have an optimal expression of the recombinant genes included in the construct, the construct may comprise further regulatory sequences, e.g., a leading DNA sequence, such as a DNA sequence derived from 5′-untranslated region (5′UTR) of a g/p gene of E. coli, a sequence for ribosomal binding. Examples of the later sequences are described in WO2020/255054A1 (incorporated herein by reference) and are illustrated in non-limiting working examples herein.

### Promoter Sequences

In the present invention, promoters may be either necessary or beneficial for achieving an optimal level of biosynthetic production of one or more HMOs in the genetically modified microorganism and allowing to achieve the desired effects according to the invention. Thus, a promoter sequence, of this invention, enables transcription and/or regulates the expression of a polypeptide capable of sugar transportation and/or the glycosyltransferases of the invention, resulting in optimized biosynthesis and transport of HMOs or HMO precursors and/or degradation of by-products of the HMO production.

In the genetically modified microorganism of the invention, a nucleic acid construct is comprised in the genetically modified microorganism, that encodes at least one gene related to biosynthetic production of one or more HMOs, a promoter DNA sequence, and other regulatory sequences, such as a ribosomal binding site sequence (e.g., Shine-Dalgarno sequence). The expression of the gene or genes related to the biosynthetic production of one or more HMOs in a genetically modified microorganism, enables production of the HMOs, making the host cell a “suitable host cell” for carrying out the invention as described. In the genetically modified microorganism, the expression, of the gene or genes related to the biosynthetic production of HMOs, as mentioned above, enables the production of one or more HMOs at level of 0.03 g/I/OD (optical density) from 1 litre of fermentation media comprising a suspension of the genetically modified microorganism s. Thus, the HMO level could be approx. 0.05 g/I/OD to approx. 0.5 g/I/OD, such as at least 0.4 g/L/OD. For the purposes of the invention, the level of HMO production is regarded as “sufficient” and the genetically modified microorganism capable of producing this level of a desired HMO or mixture of HMOs is regarded as the suitable genetically modified microorganism for carrying out the invention.

### Use

The present invention relates to a novel fermentation process of a host microorganism as described herein in the production of one or more Human Milk Oligosaccharides (HMOs). In particular, the invention relates to the fermentation process as described herein for the production of a specific HMO, wherein the host microorganism is selected with the aim of generating a majority of one specific HMO or a predominant combination of HMOs. HMO products of the invention as described are preferably 2′-FL, DFL, 2′-FL/DFL, 3′SL, 6′SL, LNnT, LNT, 3-FL, LNFP-1 and mixtures thereof, preferably selected from 2′-FL, DFL, 2′-FL/DFL, 3′-SL, 6′-SL and/or LNnT.

As is demonstrated in the experimental section, the use of the herein described fermentation process led to a 20% increase in overall productivity for MP2 and MP1 strains, and a 60% increase in productivity for MP3.

Thus, the fermentation process of the present invention enhances the amount of specific HMO product produced by at least 10, 20, 30, 40, 50 or 60%, depending on the HMO produced and the microorganism employed.

### General

It should be understood that any feature and/or aspect discussed above in connections with the described invention apply by analogy to the methods described herein.

The following figures and examples are provided below to illustrate the present invention. They are intended to be illustrative and are not to be construed as limiting in any way.

### Examples

### Materials and Methods

Unless otherwise noted, standard techniques, vectors, control sequence elements, and other expression system elements known in the field of molecular biology are used for nucleic acid manipulation, transformation, and expression. Such standard techniques, vectors, and elements can be found, for example, in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972) (Cold spring Harbor Laboratory Press, NY)

The embodiments described below are selected to illustrate the invention and are not limiting the invention in any way.

### Strains

As background strains for the strains used in the examples below the bacterial strain MDO, was used. MDO is constructed from Escherichia coli K-12 DH1. The E. coli K-12 DH1 genotype is: F⁻, λ-, gyrA96, recA1, relA1, endA1, thi-1, hsdR17, supE44. In addition to the E. coli K-12 DH1 genotype MDO has the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli is well known to the person skilled in the art.

Insertion of genetic cassettes into the E. coli chromosome can be done using gene gorging (see for example Herring and Blattner 2004 J. Bacteriol. 186: 2673-81 and Warming et al 2005 Nucleic Acids Res. 33(4): e36) with specific selection marker genes and screening methods.

The genotypes inserted into the strains used in the present application are shown in table 5, all the genes are chromosomally integrated.

TABLE 5

| Strain ID | HMO Product | Genomic Description |
|---|---|---|
| MP1 | 2′-FL/DFL mix | MDO, CA¹, futC² |
| MP2 | 2′-FL | MDO, CA¹, futC², nec³ |
| MP3 | LNnT | MDO, galT⁴, IgtA⁵, vag⁶, |
| | | lacY⁷, vgb⁸, scrYA⁹, scrBR⁹ |

¹extra colanic acid gene cluster (gmd-wcaG-wcaH-wcal-manC-manB) under the control of a PglpF promoter at a locus that is different than the native locus.
²α-1,2-fucosyltransferase from Helicobacter pylori (GenBank ID: WP_080473865.1, but with two additional amino acids (LG) at the C-terminus) inserted into chromosomal locus under control of a PglpF promoter (SEQ ID NO: 12 in WO2019/123324).
³Major Facilitator Superfamily transporter protein from Rosenbergiella nectarea (GenBank ID: WP_092672081.1) under control of a PglpF_SD7 promoter (SEQ ID NO: 19 of WO2019/123324).
⁴β-1,4-galactosyltransferase from Helicobacter pylori 26695 (GenBank ID: WP_001262061.1) under control of a PglpF promoter (SEQ ID NO: 12 in WO2019/123324).
⁵β-1,3-N-acetylglucosaminyltransferase from Neisseria meningitidis 053442 (GenBank ID: WP_002248149.1 without signal peptide) under control of a PglpF promoter (SEQ ID NO: 12 in WO2019/123324).
⁶Major Facilitator Superfamily transporter protein from Pantoea vagans (GenBank ID: WP_048785139.1) under control of a PglpF promoter (SEQ ID NO: 12 in WO2019/123324).
⁷additional copy of lactose permease from e-coli.
⁸hemoglobin from Vitreoscilla sp. HG1 (GenBank ID: AAG17874.1).
⁹PTS-dependent sucrose utilization transport system described in WO2015/197082.

### Seed Fermentation Process (Superseed) and Associated Measurements for MP1, MP2 and MP3

The seed bioreactor was filled with 27 tons of fermentation basal medium, a fully defined minimal salt medium prepared with phosphoric acid (75%), KOH solution (45%), MgSO₄, trace elements, citric acid, antifoam, and thiamine. The medium pH was controlled at 6.8 by sparging of gaseous NH₃ together with the inlet air. Ammonia also served as the only source of nitrogen. The trace elements included Mn, Cu, Fe, and Zn as sulphate salts. Lactose was not added to the seed bioreactor, as the purpose of seed fermenter is to build a biomass for the main fermenter, without HMO production. Aeration, stirring, and overpressure were applied to control dissolved oxygen at 80% of air saturation, as measured by an oxygen probe located in the lower part of the fermenter. Seed fermentation was started by inoculation from two shake flask pre-cultures with 1 L each of culture, grown in a similar basal medium, which were supplemented by 10 g of glucose/kg medium (in the following just written as g/kg [energy source]) as carbon and energy source for MP2 and MP1, and 10 g/kg of sucrose in case of MP3. The seed fermenter basal medium was supplemented with 12.3 g/kg glucose for MP2 and MP1, and 12.3 g/kg sucrose in case of MP3. Once the carbon in the basal medium was nearly consumed in the seed fermenter (end of batch phase), a continuous feed of sterile glucose solution was initiated (approximately 60% w/w) as the only source of carbon and energy at a pre-determined feeding profile (see FIG. 2A) for MP2 and MP1 (55% w/w sucrose in case of MP3 at the same feeding profile). The carbon feed was started shortly before the end of batch phase to prevent full sugar depletion. The feed was initiated manually when a carbon dioxide evolution rate (CER) of 45 was reached. The carbon feeding profile was designed to ensure carbon-limited conditions to avoid overflow metabolism and acetic acid formation. Mineral salt levels were maintained by continuously feeding a separate co-feed solution to the fermenter, containing all the constituents of the basal medium. The fermentation temperature was kept at 33° C. throughout the whole seed fermentation. End of fermentation was at approximately 53 hours. The seed fermentation was terminated once 89 g/kg of carbon source was added, ending at nearly 32 tons seed broth. The transfer to the main fermenter was triggered by increase in pH signalizing that all the sugar has been consumed. The increase in pH occurred within 1 minute after the carbon feed was stopped indicating that the feeding profile was carbon-limited, as intended. The end broth of the combined batch and fed-batch seed fermentation was used as inoculum for the main fermenter.

Throughout the seed fermentation, samples were taken in order to determine the concentration of organic acids such as acetic acid, formic acid and glutamic acid using capillary electrophoresis. Broth samples were diluted three-fold in deionized water and boiled for 20 minutes, followed by centrifugation, whereafter the resulting supernatant was analysed by capillary electrophoresis. The above measurements were used to follow the acetic acid development and to ensure that the combined batch and fed-batch seed fermentation delivered the desired acid levels. The biomass was measured using OD600, which is a common method to determine growth in the fermenter.

### Main Fermentation Process (Primary Bioreactor) and Associated Measurements MP2 and MP1

The example fermentations with strain MP2 were carried out at regular manufacturing scale, starting at a filling of approximately 86 tons (after inoculation from a seed fermenter) and ending above 300 tons of final broth. The fermentation basal medium was a fully defined minimal salt medium prepared with phosphoric acid (75%), KOH solution (45%), MgSO4, trace elements, citric acid, antifoam and thiamine. The medium pH was controlled at 6.8 by sparging of gaseous NH3 together with the inlet air. The ammonia also served as the only source of nitrogen. The trace elements included the elements Mn, Cu, Fe, Zn as sulphate salts, as well as Molybdate and Selenite. Lactose was added separately in a continuous fashion and was maintained at approximately 40 g/kg throughout most of the fermentation, until the last day, when addition stopped and the ongoing conversion to 2′-FL caused the lactose to drop to below 10 g/kg. Aeration, stirring, and overpressure were applied to control dissolved oxygen at 200% of air saturation, as measured by oxygen probes located at several places along the height of the fermenter. Fermentations were started by inoculation from a seed fermenter pre-culture, grown as described above.

In the main fermenter (primary bioreactor), a sterile glucose feed solution (approximately 60% w/w) was fed continuously as the only source of carbon and energy at a pre-determined feeding profile (see FIG. 6). The glucose feeding profile was originally designed to ensure carbon-limited conditions throughout the production phase in order to avoid overflow metabolism and acetic acid formation. Mineral salt levels were maintained by continuously feeding a separate co-feed solution to the fermenter, containing all the constituents of the basal medium. The fermentation temperature setpoint was dropped from 33° C. to 32° C. at approximately 30 h after inoculation. End-of-fermentation was at approximately 65 hours.

Throughout the fermentation, samples were taken in order to determine the concentration of 2′-FL, by-product DFL, lactose and other minor by-products using HPLC. Total broth samples were diluted three-fold in deionized water and boiled for 20 minutes, followed by centrifugation, whereafter the resulting supernatant was analysed by HPLC. The above measurements were used to accurately calculate the 2′-FL titre, the ratio of DFL/2′-FL and the accumulated yield of 2′-FL on the carbon source.

### Main Fermentation Process (Primary Bioreactor) and Associated Measurements MP3

The example fermentations with strain MP3 were carried out at regular manufacturing scale, starting at a filling of approximately 86 tons (after inoculation from a seed fermenter) and ending above 300 tons of final broth. The fermentation basal medium was a fully defined minimal salt medium prepared with phosphoric acid (75%), KOH solution (45%), MgSO4, trace elements, citric acid, antifoam and thiamine. The medium pH was controlled at 6.8 by sparging of gaseous NH3 together with the inlet air. The ammonia also served as a nitrogen source. A second nitrogen source was a continuous feed of ammonium sulphate, which was required since nitrogen is part of the LNnT molecule and therefore there is a higher demand for it. The trace elements included the elements Mn, Cu, Fe, Zn as sulphate salts, as well as Molybdate and Selenite. Lactose was added separately in a continuous fashion and was maintained at approximately 25 g/kg throughout most of the fermentation, until the last day, when addition stopped and the ongoing conversion to LNnT caused the lactose to drop to below 10 g/kg. Aeration, stirring, and overpressure were applied to control dissolved oxygen at 200% of air saturation, as measured by oxygen probes located at several places along the height of the fermenter.

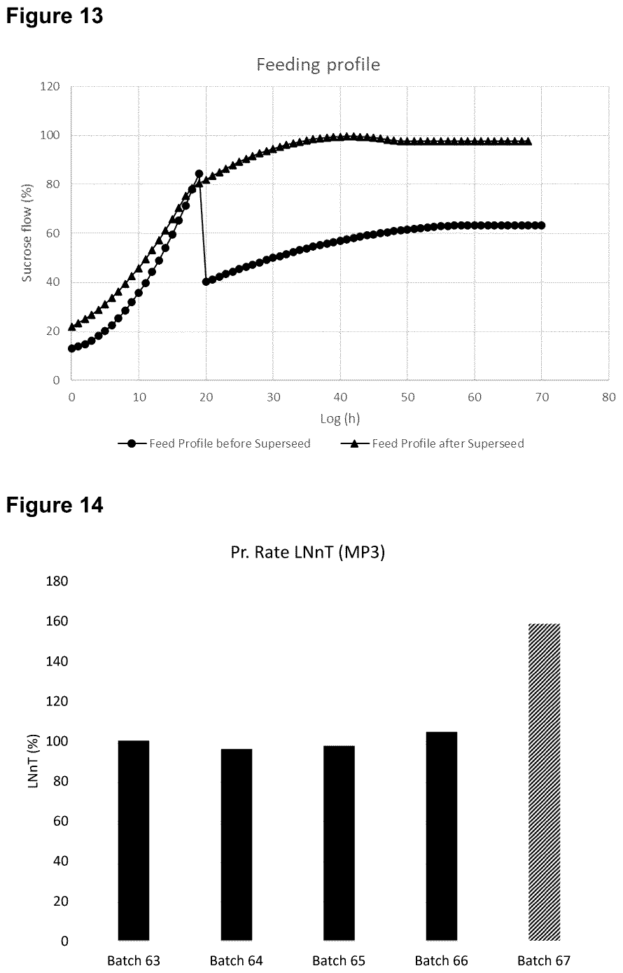

Fermentations were started by inoculation from a seed fermenter pre-culture, grown as described earlier. In the main fermenter, a sterile sucrose feed solution (approximately 55% w/w) was fed continuously as the only source of carbon and energy at a pre-determined feeding profile (see FIG. 13). The sucrose feeding profile was originally designed to ensure carbon-limited conditions throughout the production phase in order to avoid overflow metabolism and acetic acid formation. Mineral salt levels were maintained by continuously feeding a separate co-feed solution to the fermenter, containing all the constituents of the basal medium. The fermentation temperature setpoint was dropped from 33° C. to 30° C. at approximately 20 h after inoculation. End-of-fermentation was at approximately 66 hours.

Throughout the fermentation, samples were taken in order to determine the concentration of LNnT, by-products LNTII and p-LNnH, lactose and other minor by-products using HPLC. Total broth samples were diluted three-fold in deionized water and boiled for 20 minutes, followed by centrifugation, whereafter the resulting supernatant was analysed by HPLC. The above measurements were used to accurately calculate the LNnT titre, the ratio of LNTII/LNnT and p-LNnH/LNnT, and the accumulated yield of LNnT on the carbon source.

### Example 1—Testing and Implementation of Superseed on MP2 Fermentation

The present example illustrates that a combined batch and fed-batch mode in the initial phase of fermentation (seed fermenter), is capable of decreasing the acetic acid production and increase the biomass that enters the main fermentation (producing the HMO of interest).

In the production process, the seed fermenter is the second step in the fermentation process that aims at increasing biomass without any product formation.

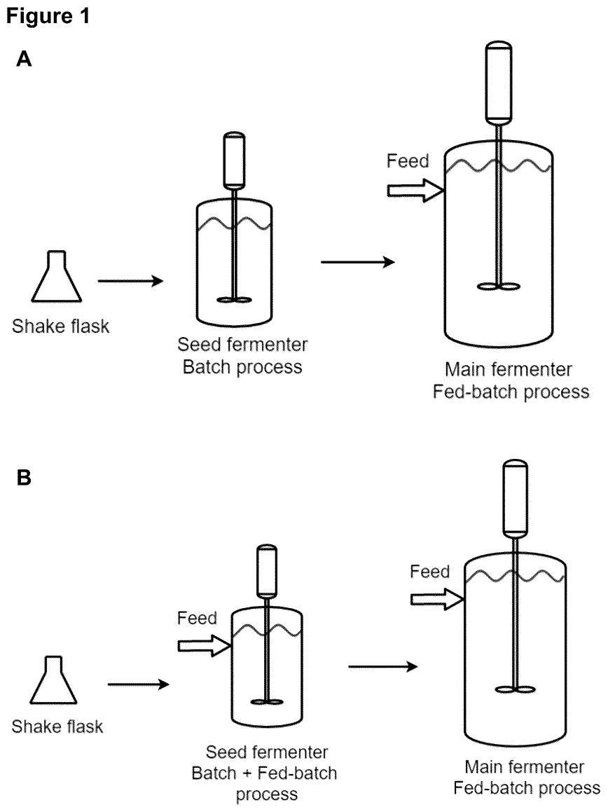

The conventional batch-only seed process, displayed in FIG. 1A, involved utilization of batch mode during seed fermentation, with use of initial 31 g of 100% glucose/kg medium, an no further additions of glucose during fermentation. Since E. coli produces significant amount of acetic acid in a batch mode, this process needs to be stopped before the concentration of acetic acid becomes toxic and affects the growth, this resulting a certain limitation in the biomass that can be achieved in the seed fermenter.

FIG. 1B shows the concept of the current invention, where the fed-batch operation works as an extension of the conventional seed fermentation. In the combined batch and fed-batch seed fermentation concept (superseed), the process starts as in the conventional batch seed process, but with lower amount of sugar. (12.3 g of 100% glucose/kg medium). Once the sugar has been consumed, a continuous sugar feed is supplied to further increase the biomass to the desired level in a controlled manner. The feed was started shortly before the sugar was fully consumed as described in the “Seed fermentation process (superseed)” in the “Materials and method” section above. The sugar feed continued until 93 g of 100% glucose/kg of start medium had been added to the seed fermenter.

The relative glucose feeding profile utilized in the combined batch and fed-batch seed fermentation is shown in FIG. 2A in black solid line. With this profile, the expected biomass development can be seen in FIG. 2B, with growth rate μ=0.09 h\hat{ }(−1). The growth rate was calculated using biomass development measured in the conventional batch seed fermenter on batch 2FL103 in the exponential growth phase. FIG. 2A further shows three additional feeding profiles represented in dashed lines that could be utilized in the combined batch and fed-batch seed fermentation if it was desired to run the process faster, with corresponding biomass developments displayed in FIG. 2B. The starting level of glucose feeding was determined by the biomass-specific sugar feed rate, also known as qS. The starting qS was set to 60, which is a well-known level where the cells are able to grow and consume acetic acid at the same time. The glucose feed at the start of feeding (t0) was calculated using the below equation.

$$carbon {flow}_{{t}_{0}} = {q}_{S} \times {biomass}_{{t}_{0}}$$

$${biomass}_{{t}_{0}} = \frac{{OD}_{{t}_{0}} \times {V}_{{t}_{0}}}{{10}^{6}}$$

The biomass was calculated as OD multiplied by volume of the tank and divided by 10\hat{ }6

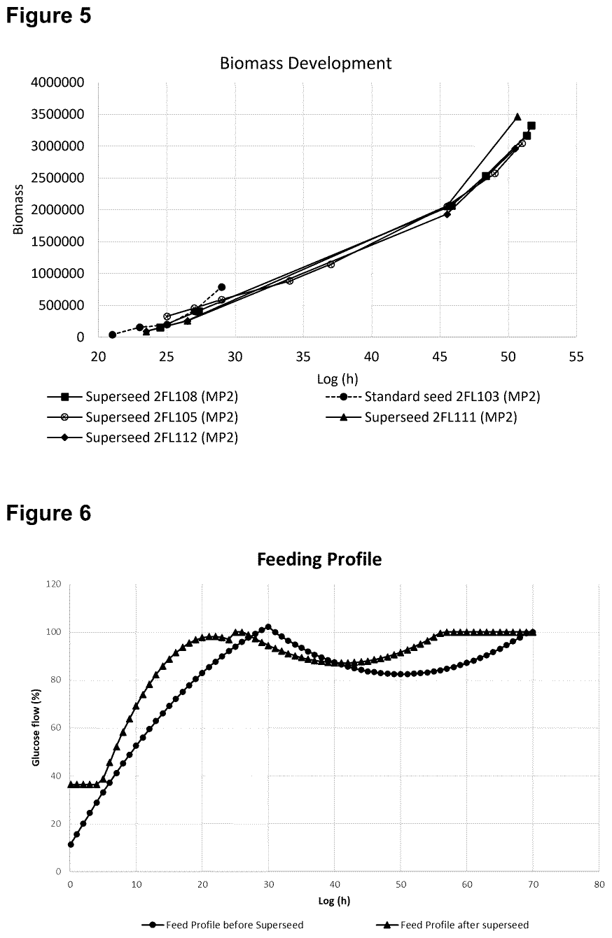

A comparison between the conventional batch-only seed process and the combined batch and fed-batch seed fermentation in terms of acetic acid, OD, and biomass development is displayed in FIG. 3, FIG. 4, and FIG. 5, respectively. Biomass was calculated as OD multiplied by the tank volume in kilograms.

As a result of better controlled growth through continuous feed, the combined batch and fed-batch seed fermentation resulted in 30 times reduced acetic acid and more than 3 times higher biomass in comparison to the conventional batch seed fermentation process. The significant increase in biomass in the batch and fed-batch seed fermenter also leads to higher biomass inoculum of the main fermenter producing the HMO, where the conventional batch seed fermenter delivered a biomass of 7.9E+05 while the combined batch and fed-batch seed fermentation delivers a biomass of 3.2E+06. Furthermore, as FIG. 4 reveals, the OD development in all four of the combined batch and fed-batch seed fermentation runs followed the expected OD development that was illustrated in FIG. 2B and calculated based on the feeding profile. The latter shows that the combined batch and fed-batch seed fermentation is a well-controlled process.

The combined batch and fed-batch seed fermentation provided controlled growth of bacteria through fed-batch mode, thus preventing the accumulation of acetic acid, as was shown in FIG. 3, this allowed the cells to enter the main fermentation in a better condition, since accumulation of acetic acid can stress the cells and have a negative impact on the growth.

The higher biomass originating from the combined batch and fed-batch seed fermentation (FIG. 5) allowed an increased sugar feed in the main fermentation thus reaching higher productivity and high yield faster. FIG. 6 shows the relative glucose feed in main fermenter using the conventional batch seed process and the combined batch and fed-batch seed fermentation. As FIG. 6 reveals, the combined batch and fed-batch seed fermentation allows using higher sugar feed at the beginning of main fermentation, which allows higher overall productivity in the main fermenter. FIG. 7 shows the overall relative production rate using the conventional batch seed and the combined batch and fed-batch seed fermentation for 2′-FL fermentation using MP2 strain grown on glucose. The combined batch and fed-batch seed fermentation batches resulted on average in 20% increase in overall productivity

### Example 2—Testing and Implementation of Superseed on MP1

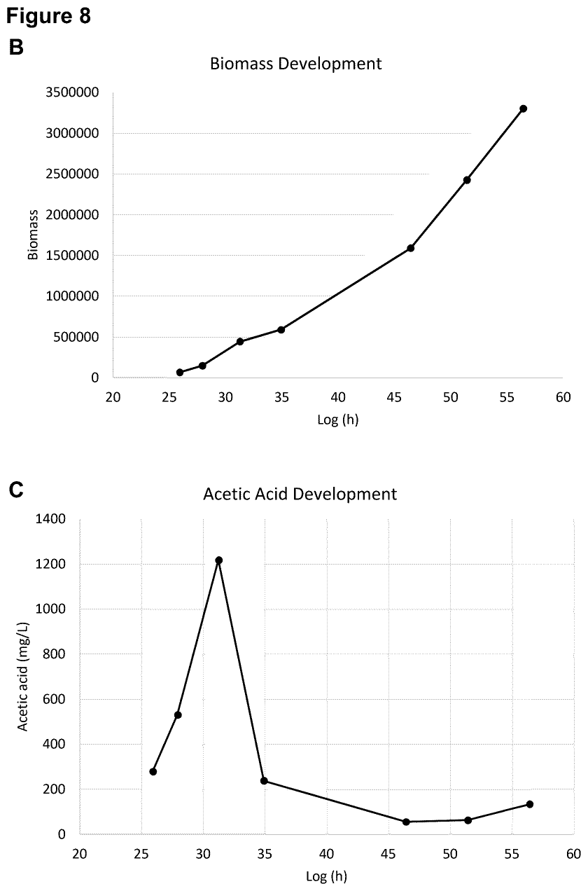

The same superseed and main fermentation process as described in example 1 was implemented on strain MP1 producing 2′-FL/DFL. FIGS. 8 (A), (B) and (C) show development of OD, biomass and acetic acid, respectively for batch 2FL140. All three parameters show the same pattern as previously shown on numerous MP2 superseed runs.

The only difference between the MP2 and MP1 superseed fermentations was the length of the initial batch phase, before the start of feed. The growth of bacteria in batch culture can be modeled with four different phases: lag phase, log phase also known as exponential phase, stationary phase, and death phase. The period of little to no cell division is called the lag phase and can last for 1 hour to several days. During lag phase, bacteria adapt themselves to the growth conditions. The lag phase of the MP2 and MP1 strains differed, where the MP1 had a lag phase 4 hours longer then the MP2 strain. As the superseed concept involves controlled growth and reduced acid production at a predetermined and well-defined feed rate, the length of the lag phase wherein the bacteria growth is uncontrolled in not relevant when assessing the efficiency of the fed-batch phase.

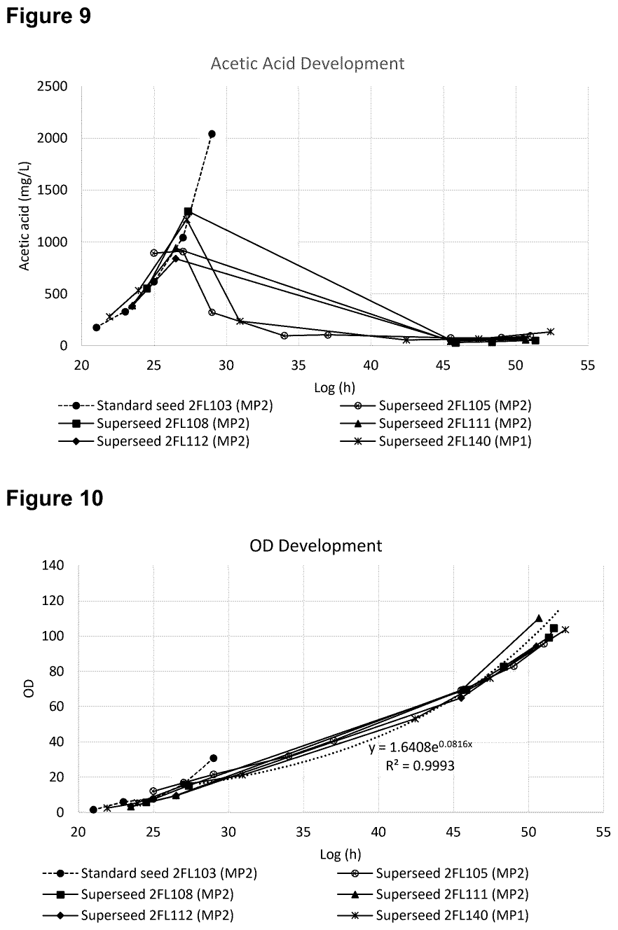

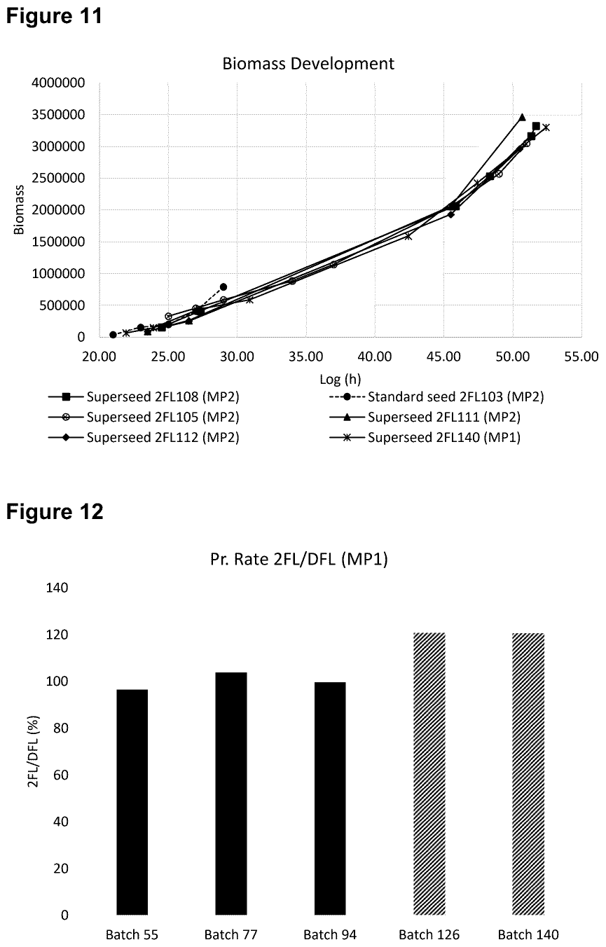

FIGS. 9, 10, and 11 show the OD, biomass and acid development for the MP2 superseed fermentations (already presented in FIGS. 3, 4 and 5) in the same graph as the MP1 superseed fermentation (already presented in FIGS. 8 A, B and C). In order to compare the developments of these three parameters between MP2 and MP1 during the fed-batch phase, the 4 hours difference in lag phase has been subtracted from MP1 data. FIGS. 9, 10 and 11 show that MP1 delivered results withing the same expected range as MP2, illustrating the robustness of the superseed concept.

The overall relative production rate using the conventional batch seed fermentation and the combined batch and fed-batch seed fermentation for 2′-FL/DFL fermentation using MP1 strain grown on glucose is shown in FIG. 12. The combined batch and fed-batch seed fermentation batches resulted on average in 20% increase in overall productivity in the main fermentation, which is the same as was achieved with the MP2 strain (FIG. 7).

### Example 3—Testing and Implementation of Superseed on MP3

The same superseed process as described in the previous two examples was implemented on strain MP3 producing LNnT, with the difference in carbon source, in that MP3 was grown on sucrose. The main fermentation process was different in comparison to MP2 and MP1, as described in the “Materials and methods” section above. FIG. 13 shows the relative sucrose feed in main fermenter using the conventional batch seed process and the combined batch and fed-batch seed fermentation. For the primary fermenter seeded with the combined batch and fed-batch seed fermentation, the sucrose feed was initiated at 270 kg/h. Similarly, as it was the case for MP2 and MP1, the combined batch and fed-batch seed fermentation allows using higher sugar feed at the beginning of the main fermentation, which allows higher overall productivity in the main fermenter.

The overall relative production rate using the convention batch seed fermentation and the combined batch and fed-batch seed fermentation for LNnT fermentation using MP3 strain grown on sucrose is shown in FIG. 14. The combined batch and fed-batch seed fermentation batches resulted on average in 60% increase in overall productivity, which was even higher than what was observed with the MP2 and MP1 stains in example 1 and 2.

Another benefit of the combined batch and fed-batch seed fermentation was observed in LNnT produced from MP3 fermentation. The two most abundant side-products in LNnT fermentation are para-lacto-N-neohexaose (p-LNnH) and lacto-N-triose II (LNT2). For an LNnT product the side-product should preferably be below 20% for p-LNnH and below 9% for LNT2, and it is desired to end the fermentation with as low a side-product ratio as possible. As revealed in FIG. 15, the implementation of the combined batch and fed-batch seed fermentation resulted in significant decrease in impurities with 30% decrease in p-LNnH and 35% decrease in LNT2 ratio relative to LNnT.

### CONCLUSION

In the current combined batch and fed-batch seed fermentation process the higher biomass allows application of increased carbon source feed in the main fermentation, thus reaching the area of high productivity faster as well as increasing the overall productivity by 20% or more.

There are at least two factors in the combined batch and fed-batch seed fermentation that leads to this.

The acetic acid level in the seed fermenter is below 200 mg/L when used for seeding the main fermenter as compared to the conventional batch seed fermentation where the acetic acid is above 2000 mg/L. The low level of acidic acid at the time of seeding results in healthier cells in the main fermenter, preventing a lag phase after seeding.

Secondly, the combined batch and fed-batch seed fermentation results in more than 3 times increase of biomass, which allows the feeding of the main fermenter to start at a higher carbon source flow.

A further potential advantage of the combined batch and fed-batch seed fermentation process is that it is a well-controlled and since there is more space in the seed fermenter, the biomass could be increased even further. Therefore, the combined batch and fed-batch seed fermentation could be used to seed two production fermenters, instead of just one as done with the conventional batch seed fermentation. This essentially allows the operation of two or more main fermenters at the same time, with only one seed fermenter available to seed them, thereby increasing production efficiency even further.

1. A fermentation process for producing one or more Human Milk Oligosaccharides (HMOs) comprising,
a) providing a seed bioreactor with one or more feed lines, wherein the seed bioreactor contains a liquid medium comprising no more than 5-40 g of a carbon source/kg of medium
b) inoculating the seed bioreactor with HMO producing microorganisms,
c) operating the seed bioreactor at conditions to promote growth of the microorganisms by continuously feeding to the seed bioreactor a medium with one or more carbon sources,
d) providing a primary bioreactor containing a liquid medium capable of supporting growth of the microorganisms,
e) passing at least a portion of the microorganism culture from the seed bioreactor, into the primary bioreactor,
f) operating the primary bioreactor at conditions to promote growth of the microorganisms and to promote HMO production from the microorganisms, while continuously feeding to the primary bioreactor a medium with one or more carbon sources and continuously adding to the primary bioreactor a substrate for the HMO production,
g) fermenting the added carbon sources and substrate to produce a fermentation broth comprising HMO producing microorganisms and one or more HMO products and optionally,
h) harvesting or purifying the one or more HMOs from the fermentation broth.

2. The fermentation process according to claim 1, wherein the seed bioreactor contains a liquid medium comprising no more than 13 g of a carbon source/kg of medium.

3. The fermentation process according to claim 1, wherein the substrate for the HMO production continuously added to the primary bioreactor is selected from the group consisting of lactose, a fucosylated N-acetyl-glucosaminylated lactose trisaccharide, a sialylated N-acetyl-glucosaminylated lactose trisaccharide, and an N-acetyl-glucosaminylated lactose trisaccharide.

4. The fermentation process according to claim 1, wherein the continuous feeding in step c) is initiated when the carbon source added in step a) is close to depletion.

5. The fermentation process according to claim 1, wherein the liquid medium in the primary fermenter in step d) does not comprise a carbon source.

6. The fermentation process according to claim 1, wherein the fermentation conditions are aerobic in both the seed and primary bioreactor.

7. The fermentation process according to claim 1, wherein the feeding of step c) and/or step f) comprises stepwise addition of one or more carbon sources or follows a linear and/or non-linear feeding profile of one or more carbon sources, or a combination thereof.

8. The fermentation process according to claim 1, wherein the feeding medium used in step c) does not contain a substrate for HMO production, wherein the substrate is lactose, or a fucosylated, sialylated or N-acetyl-glucosaminylated lactose trisaccharide or tetrasaccharide.

9. The fermentation process according to claim 1, wherein the continuously feeding to the seed bioreactor or primary bioreactor of one or more carbon sources results in a reduced acetic acid formation in the fermentation broth.

10. The fermentation process according to claim 1, wherein the continuously feeding to the seed bioreactor of one or more carbon sources results in an acetic acid formation below 250 mg/L, such as below 100 mg/L in the seed culture at the end of fermentation, as measured by capillary electrophoresis.

11. The fermentation process according to claim 1, wherein the seed bioreactor in step c) is run for at least 30 hours.

12. The fermentation process according to claim 1, wherein primary bioreactor is initially feed with a 10-20 g of a carbon source/kg of medium in step f).

13. The fermentation process according to claim 1, wherein HMO formation in the primary bioreactor is increased by at least 10% when using the seed culture generated in step a)-c) for seeding the primary bioreactor (step e) compared to using an un-fed batch culture to seed the primary bioreactor.

14. The fermentation process according to claim 1, wherein the one or more carbon source(s) is/are selected from the group consisting of glycerol, glucose, sucrose and mixtures thereof.

15. The fermentation process according to claim 1, wherein HMO producing the microorganism is selected form the group consisting of Escherichia coli, Saccharomyces cerevisiae, Yarrowia lipolytica, Schizosaccharomyces pombe, Pichia pastoris, Kluveromyces lactis and Kluveromyces marxianus.

16. The fermentation process according to claim 1, wherein the HMO producing microorganisms express one or more proteins enabling the production of one or more HMOs in the cell and wherein the expression of the one or more proteins is controlled by one or more genetic regulatory elements.

17. The fermentation process according to claim 16, wherein the one or more genetic regulatory elements regulates the expression of the protein through the concentration of the one or more carbon sources.

18. The fermentation process according to claim 16, wherein the one or more genetic regulatory elements comprises a Plac or PglpF promoter sequence or a functional variant thereof.

19. The fermentation process according to claim 1, wherein the one or more HMOs produced are selected from the group consisting of LNT-II, pLNnH, LNT, LNnT, LNFP-I, LNFP-II, LNFP-III, LNFP-V, LNFP-VI, LNDFH-I, LNDFH-II, LNDFH-III, 2′-FL, DFL, 3FL, LST-a, 3′SL, 6′SL, LST-b, LST-c, FSL, FLST-a, DSLNT, LNnH and LNH.

20. The fermentation process according to claim 1 wherein the seed bioreactor or primary bioreactor comprises one or more control units selected from the group consisting of a temperature control unit, an aeration control unit, a growth rate control unit, a biomass control unit, an acetic acid control unit, a feed rate control unit, a titer rate control unit, an overpressure control unit and a pH control unit.
